(19) United States
(12) Patent Application Publication
(10) Pub. No.: US 2019/0076242 A1
(43) Pub. Date: Mar. 14, 2019

(54) METHODS OF PROVIDING EXTENDED DEPTH OF FIELD AND/OR ENHANCED DISTANCE VISUAL ACUITY

(71) Applicant: Candido Dionisio PINTO, Monrovia, CA (US)

(72) Inventor: Candido Dionisio PINTO, Monrovia, CA (US)

(21) Appl. No.: 16/126,806

(22) Filed: Sep. 10, 2018

Related U.S. Application Data
(60) Provisional application No. 62/556,304, filed on Sep. 8, 2017.

(51) Int. Cl. A61F 2/16

(52) U.S. Cl. CPC A61F 2/1651; A61F 2002/169; A61F 2/164; A61F 2/1618

(57) ABSTRACT

Methods of implanting a first artificial lens into an eye of a human can include inserting the first artificial lens anterior of a second artificial lens. At least one of the first and second lenses can include an optic and one or more haptic portions disposed about the optic. The optic can include transparent material. The optic can have an anterior surface and a posterior surface. At least one of the anterior and posterior surfaces can include an aspheric surface.

### CROSS REFERENCE TO RELATED APPLICATIONS

This application claims priority to U.S. Provisional Application No. 62/556,304 filed Sep. 8, 2017 which is herein incorporated by reference in its entirety.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

This disclosure relates to methods of using ophthalmic implants, for example, ophthalmic implants with extended depth of field. FIG. 1 is a schematic illustration of the human eye. As shown in FIG. 1, the human eye 100 includes a cornea 110, an iris 115, a natural crystalline lens 120, and a retina 130. Light enters the eye 100 through the cornea 110 and towards the pupil, which is the opening in the center of the iris 115. The iris 115 and pupil help regulate the amount of light entering the eye 100. In bright lighting conditions, the iris 115 closes the pupil to let in less light, while in dark lighting conditions, the iris 115 opens the pupil to let in more light. Posterior to the iris 115 is a natural crystalline lens 120. The cornea 110 and the crystalline lens 120 refract and focus the light toward the retina 130. In an eye 100 with a visual acuity of 20/20, the crystalline lens 120 focuses the light to the back of the eye onto the retina 130. The retina 130 senses the light and produces electrical impulses, which are sent through the optic nerve 140 to the brain. When the eye does not properly focus the light, corrective and/or artificial lenses have been used.

### SUMMARY OF THE DISCLOSURE

Certain embodiments described herein include a lens configured for implantation into an eye of a human. The lens can include an optic comprising transparent material. The lens can also include haptic portions disposed about the optic to affix the optic in the eye when implanted therein. The optic can include an anterior surface and a posterior surface. The anterior surface can be convex and the posterior surface can be concave such that the optic is meniscus shaped. Each of the convex anterior surface and the concave posterior surface can have a surface vertex. The optic can have an optical axis through the surface vertices. In various embodiments, a thickness along the optical axis can be between about 100-700 micrometers (or any range formed by any of the values in this range). In addition, the anterior and posterior surfaces can comprise aspheric surfaces.

Certain embodiments described herein include a lens configured for implantation into an eye of a human. The lens can include an optic comprising transparent material. The lens can also include at least one haptic disposed with respect to the optic to affix the optic in the eye when implanted therein. The optic can have an anterior surface and a posterior surface. The anterior surface can be convex and the posterior surface can be concave such that the optic is meniscus shaped. Each of the convex anterior surface and the concave posterior surface can have a surface vertex. The optic can have an optical axis through the surface vertices. In various embodiments, the anterior and posterior surfaces can comprise aspheric surfaces. The anterior surface can have an aspheric shape that comprises a conic or biconic offset by perturbations comprising an aspheric higher order function of radial distance from the optical axis.

In some such embodiments, the aspheric higher order function can include at least one even order term, a₂ₙr²ⁿ, where n is an integer and a₂ₙ is a coefficient and r is the radial distance from the optical axis. For example, the aspheric higher order function can include a second order term, a₂r², where a₂ is a coefficient and r is the radial distance from the optical axis. As another example, the aspheric higher order function can include a fourth order term, a₄r⁴, where a₄ is a coefficient and r is the radial distance from the optical axis. The aspheric higher order function also can include a sixth order term, a₆r⁶ where a₆ is a coefficient and r is the radial distance from the optical axis. Furthermore, the aspheric higher order function can include an eighth order term, a₈r⁸ where a₈ is a coefficient and r is the radial distance from the optical axis. In some embodiments of the lens, the optic can have a thickness along the optical axis that is between about 100-700 microns (or any range formed by any of the values in this range). In various embodiments, the anterior surface has an aspheric shape that comprises a biconic offset by the perturbations.

Certain embodiments described herein include a lens configured for implantation into an eye of a human. The lens can include an optic comprising transparent material. The lens can also include at least one haptic disposed with respect to the optic in the eye when implanted therein. The optic can have an anterior surface and a posterior surface. The anterior surface can be convex and the posterior surface can be concave such that the optic is meniscus shaped. Each of the convex anterior surface and the concave posterior surface can have a surface vertex. The optic can have an optical axis through the surface vertices. In various embodiments, the anterior and posterior surfaces can comprise aspheric surfaces. The posterior surface can have an aspheric shape that comprises a conic or biconic offset by perturbations comprising an aspheric higher order function of radial distance from the optical axis. In various embodiments, the posterior surface has an aspheric shape that comprises a biconic offset by the perturbations.

Certain embodiments described herein include a lens configured for implantation into an eye of a human. The lens can include an optic comprising transparent material. The optic can have an anterior surface and a posterior surface. The anterior surface can comprise an aspheric surface. The anterior and posterior surfaces also can be shaped to provide average modulation transfer function (MTF) values that are between 0.1 and 0.4 at 100 lines per millimeter for at least 90% of the object vergences within the range of 0 to 2.5 Diopter (D) when the optic is inserted into the human eye having an aperture size of aperture size of 2 to 6 millimeters, 3 to 6 millimeters, or 4 to 6 millimeters (e.g., the aperture size can be 2 mm, 3 mm, 4 mm, 6 mm, any value within these ranges, or any range formed by such values). The average MTF values can comprise MTF values at 100 lines per millimeter integrated over the wavelengths between about 400 to 700 nm weighted by the photopic luminosity function for on axis objects.

In various embodiments, the human eye comprises a crystalline lens and the average modulation transfer function values are provided when the optic is inserted anterior of the crystalline lens. In various other embodiments, the human eye excludes a crystalline lens and the modulation transfer function values are provided when the optic is inserted in place of the crystalline lens. The lens further can comprise haptic portions. In addition, the optic can have an optical axis and a thickness through the optical axis that is between about 100-700 microns (or any range formed by any of the values in this range).

Certain embodiments described herein include a lens configured for implantation into an eye of a human. The lens can include an optic comprising transparent material. The optic can have an anterior surface and a posterior surface. The anterior surface can comprise an aspheric surface. The anterior and posterior surfaces also can be shaped to provide average modulation transfer function (MTF) values that are between 0.1 and 0.4 at 100 lines per millimeter for at least 90% of the object vergences within the range of 0 to 2.5 Diopter (D) when the optic is inserted into a model eye having an aperture size of 2 to 6 millimeters, 3 to 6 millimeters, or 4 to 6 millimeters (e.g., the aperture size can be 2 mm, 3 mm, 4 mm, 6 mm, any value within these ranges, or any range formed by such values). The average MTF values can comprise MTF values at 100 lines per millimeter integrated over the wavelengths between about 400 to 700 nm weighted by the photopic luminosity function for on axis objects.

The model eye can comprise a Liou-Brennan model eye. Alternatively, the model eye can comprise a Badal model eye. Furthermore, the model eye can comprise an Arizona model eye or an Indiana model eye. Other standardized or equivalent model eyes can be used.

In some embodiments, the modulation transfer function values can be provided when the optic is inserted in the model eye in a phakic configuration. In some other embodiments, the modulation transfer function values can be provided when the optic is inserted in the model eye in an aphakic configuration. The lens can further comprise haptic portions. Furthermore, the optic can have an optical axis and a thickness through the optical axis that is between about 100-700 microns (or any range formed by any of the values in this range).

Certain embodiments described herein include a lens configured for implantation into an eye of a human. The lens can include an optic comprising transparent material. The optic can have an anterior surface and a posterior surface and an exit pupil. The anterior surface can comprise an aspheric surface. The anterior and posterior surfaces can be shaped to provide a radial power profile characterized by Φ(r)=a+br²+cr⁴+dr⁶+er⁸ for wavefront at the exit pupil of the optic for an object vergence of 0 to 2.5 Diopter (D) where r is the radial distance from an optical axis extending through the surface vertices on the anterior and posterior surfaces and a, b, c, d, and e are coefficients.

Certain embodiments described herein include a lens configured for implantation into an eye of a human. The lens can include an optic comprising transparent material. The lens can also include at least one haptic disposed with respect to the optic to affix the optic in the eye when implanted therein. The optic can include an anterior surface and a posterior surface. Each of the anterior surface and the posterior surface can have a surface vertex. The optic can have an optical axis through the surface vertices. The thickness along the optical axis can be between about 100-400 micrometers (or any range formed by any of the values in this range). In addition, at least one of the anterior and posterior surfaces can comprise aspheric surfaces. In some embodiments, the anterior surface can be convex. In addition, the posterior surface can be concave.

Certain embodiments described herein include a lens configured for implantation into an eye of a human. The lens can include an optic comprising transparent material. The lens can also include at least one haptic disposed with respect to the optic to affix the optic in the eye when implanted therein. The optic can include an anterior surface and a posterior surface. Each of the anterior surface and the posterior surface can have a surface vertex. The optic can have an optical axis through the surface vertices. At least one of the anterior and posterior surfaces can comprise an aspheric surface including perturbations comprising an aspheric higher order function of radial distance from the optical axis and at least one of the surfaces can have an aspheric shape that comprises a biconic. In some embodiments, the anterior surface can be convex. In addition, the posterior surface can be concave.

Certain embodiments described herein include a lens configured for implantation into an eye of a human. The lens can include an optic comprising transparent material. The lens can also include haptic portions disposed about the optic to affix the optic in the eye when implanted therein. The optic can include an anterior surface and a posterior surface. Each of the anterior surface and the posterior surface can have a surface vertex. The optic can have an optical axis through the surface vertices. The thickness along the optical axis can be between about 100-700 micrometers (or any range formed by any of the values in this range). In addition, the anterior and posterior surfaces can comprise aspheric surfaces.

Certain embodiments described herein include a lens configured for implantation into an eye of a human. The lens can include an optic comprising transparent material. The lens can also include at least one haptic disposed with respect to the optic to affix the optic in the eye when implanted therein. The optic can include an anterior surface and a posterior surface. Each of the anterior surface and the posterior surface can have a surface vertex. The optic can have an optical axis through the surface vertices. At least one of the anterior and posterior surfaces can comprise an aspheric surface that comprises a conic or biconic offset by perturbations comprising an aspheric higher order function of radial distance from the optical axis.

In various embodiments of the lens described herein comprising a transparent material, the transparent material can comprise collamer. The transparent can comprise silicone, acrylics, or hydrogels. The transparent material can comprise hydrophobic or hydrophilic material.

In various embodiments of the lens described herein, the anterior surface can be rotationally symmetric. The anterior surface can have a shape that includes a conic or biconic term. The anterior surface can have a shape that includes a conic or biconic term and aspheric higher order perturbation terms. In some embodiments of the lens, the posterior surface can have a shape that includes a conic or biconic term. The conic or biconic term can have a conic constant having a magnitude greater than zero. For example, the conic or biconic term can have a conic constant having a magnitude of at least one. As another example, the conic or biconic term can have a conic constant having a magnitude of at least ten.

In various embodiments of the lens described herein, the posterior surface can be rotationally non-symmetric. The posterior surface can have different curvature along different directions through the optical axis of the optic. For example, the posterior surface can have different curvature along orthogonal directions through the optical axis of the optic. The shape of the posterior surface can include a biconic term. The biconic term can have a conic constant having a magnitude greater than zero. For example, the biconic term can have a conic constant having a magnitude of at least one. As another example, the conic or biconic term can have a conic constant having a magnitude of at least ten. In various embodiments of the lens described herein, the optic can have a thickness along the optical axis of between 100-400 micrometers. For example, the thickness along the optical axis can be between 100-300 micrometers, between 100-200 micrometers, between 200-300 micrometers, between 300-400 micrometers, or any range formed by any of the values in these ranges.

In various embodiments of the lens described herein, the anterior and posterior surfaces of the lens can be shaped to provide average modulation transfer function (MTF) values that are between 0.1 and 0.4 at 100 lines per millimeter for at least 90% of the object vergences within the range of 0 to 2.5 Diopter (D) when the optic is inserted into a model eye having an aperture size of 2 to 6 millimeters, 3 to 6 millimeters, or 4 to 6 millimeters (e.g., the aperture size can be 2 mm, 3 mm, 4 mm, 6 mm, any value within these ranges, or any range formed by such values). The average MTF values can comprise MTF values at 100 lines per millimeter integrated over the wavelengths between about 400 to 700 nm weighted by the photopic luminosity function for on axis objects. The model eye can comprise a Liou-Brennan model eye, a Badal model eye, an Arizona model eye, an Indiana model eye, or any standardized or equivalent model eye.

In some such embodiments, the anterior and posterior surfaces of the lens are shaped to provide average modulation transfer function (MTF) values that are between 0.1 and 0.4 at 100 lines per millimeter for at least 95% or 98% of the object vergences within the range of 0 to 2.5 Diopter (D).

In various embodiments of the lens described herein, the anterior and posterior surfaces can be shaped to provide modulation transfer functions (MTF) without phase reversal for at least 90% of the object vergences within the range of 0 to 2.5 Diopter (D) when the optic is inserted into the model eye. In some such embodiments, the anterior and posterior surfaces are shaped to provide modulation transfer functions (MTF) without phase reversal for at least 95%, 98%, 99%, or 100% of the object vergences within the range of 0 to 2.5 Diopter (D) when said optic is inserted into the model eye.

In various embodiments of the lens described herein, the anterior surface can have a radius of curvature between 0 to 1 mm, between 1×10⁻⁶ to 1×10⁻³ mm, or between 5×10⁻⁶ to 5×10⁻⁴ mm. The anterior surface can have a conic constant between −1×10⁶ to −100 or between −3×10⁵ to −2×10⁵. The posterior surface can have a radius of curvature, R_{y}, between 0 to 20 mm. The posterior surface can have a radius of curvature, Rₓ, between 0 to 20 mm. The posterior surface can have a conic constant, k_{y} between −20 to 20. The posterior surface can have a conic constant, kₓ, between −25 to 0.

In some embodiments of the lens described herein, the lens can be configured to be disposed anterior to the natural lens of the eye. In some other embodiments of the lens, the lens can be configured to be disposed in the capsular bag.

Certain embodiments described herein include a method of implanting the lens of any of the embodiments of the lens. The method can include forming an opening in tissue of the eye and inserting the lens anterior of the natural lens of the eye. Certain embodiments described herein also include a method including forming an opening in tissue of the eye and inserting the lens in the capsular bag.

In various embodiments of the lens described herein, the optic can have a thickness along the optical axis that is between about 700 microns-4 millimeter. For example, the thickness along the optical axis can be between about 700 microns-3 millimeter, between about 700 microns-2 millimeter, between about 700 microns-1 millimeter, or any range formed by any of the values in these ranges.

Certain embodiments described herein include a lens pair configured for implantation into a pair of left and right eyes of a human. The lens pair includes a first lens. The first lens can include an optic comprising transparent material. The optic of the first lens can have an anterior surface and a posterior surface. The anterior surface can include an aspheric surface. The anterior and posterior surfaces of the first lens can be shaped to provide average modulation transfer function (MTF) values that are between 0.1 and 0.4 at 100 lines per millimeter for at least 90% of the object vergences within the range of 0 to 2.0 Diopter or 0 to 2.5 Diopter (D) when the optic of the first lens is inserted into a model eye having an aperture size of 2 to 6 millimeters, 3 to 6 millimeters, or 4 to 6 millimeters (e.g., the aperture size can be 2 mm, 3 mm, 4 mm, 6 mm, any value within these ranges, or any range formed by such values). The average MTF values of the first lens can comprise MTF values at 100 lines per millimeter integrated over the wavelengths between about 400 to 700 nm weighted by the photopic luminosity function for on axis objects.

The lens pair also includes a second lens. The second lens can include an optic comprising transparent material. The optic of the second lens can have an anterior surface and a posterior surface. The anterior surface can include an aspheric surface. The anterior and posterior surfaces of the second lens can be shaped to provide average modulation transfer function (MTF) values that are between 0.1 and 0.4 at 100 lines per millimeter for at least 90% of the object vergences within the range of −2.0 to 0 Diopter or −2.5 to 0 Diopter (D) when the optic of the second lens is inserted into a model eye having an aperture size of 2 to 6 millimeters, 3 to 6 millimeters, or 4 to 6 millimeters (e.g., the aperture size can be 2 mm, 3 mm, 4 mm, 6 mm, any value within these ranges, or any range formed by such values). The average MTF values of the second lens can comprise MTF values at 100 lines per millimeter integrated over the wavelengths between about 400 to 700 nm weighted by the photopic luminosity function for on axis objects.

The model eye can comprise a Liou-Brennan model eye. Alternatively, the model eye can comprise a Badal model eye. Furthermore, the model eye can comprise an Arizona model eye or an Indiana model eye. Other standardized or equivalent model eyes can be used.

In various embodiments of the lens pair, the modulation transfer function values of the first or second lens can be provided when the optic of the first or second lens is inserted in the model eye in a phakic configuration. In various other embodiments, the modulation transfer function values of the first or second lens can be provided when the optic of the first or second lens is inserted in the model eye in an aphakic configuration.

In various embodiments of the lens pair, the first or second lens can further comprise haptic portions. The optic of the first or second lens can have an optical axis and a thickness through the optical axis that is between about 100-700 microns. In other embodiments, the optic of the first or second lens can have an optical axis and a thickness through the optical axis that is between about 700 microns-4 millimeter. In some such embodiments, the thickness along the optical axis can be between about 700 microns-3 millimeter, between about 700 microns-2 millimeter, between about 700 microns-1 millimeter, or any range formed by any of the values in these ranges.

In various embodiments of the lens pair, the anterior and posterior surfaces of the first lens can be shaped to provide average modulation transfer function (MTF) values that are between 0.1 and 0.4 at 100 lines per millimeter for at least 95% or 98% of the object vergences within the range of 0 to 2.5 Diopter (D).

In various embodiments of the lens pair, the anterior and posterior surfaces of the second lens can be shaped to provide average modulation transfer function (MTF) values that are between 0.1 and 0.4 at 100 lines per millimeter for at least 95% or 98% of the object vergences within the range of −2.5 to 0 Diopter (D).

In various embodiments of the lens pair, the anterior and posterior surfaces of the first lens can shaped to provide modulation transfer functions (MTF) without phase reversal for at least 90%, 95%, 98%, 99%, or 100% of the object vergences within the range of 0 to 2.5 Diopter (D) when said optic is inserted into the model eye.

In various embodiments of the lens pair, the anterior and posterior surfaces of the second lens can be shaped to provide modulation transfer functions (MTF) without phase reversal for at least 90%, 95%, 98%, 99%, or 100% of the object vergences within the range of −2.5 to 0 Diopter (D) when said optic is inserted into the model eye.

Certain embodiments described herein include a lens configured for implantation into an eye of a human. The lens can include an optic comprising transparent material. The optic can have an anterior surface and a posterior surface. Each of the anterior surface and the posterior surface can have a surface vertex. The optic can have an optical axis through the surface vertices. At least one of the anterior and posterior surfaces can comprise a surface having a first portion and a second portion. The first portion can be disposed centrally about the optical axis. The second portion can surround the first portion and can have a different surface profile than the first portion. The first portion can be configured to provide an extended depth of field. The second portion can be configured to provide an enhanced vision quality metric at distance in comparison to the first portion.

In some such embodiments, distance can comprise objects between infinity to 2 meters or distance can comprise 0 D vergence. In various embodiments of the lens, the lens can further comprise a third portion surrounding the second portion. The third portion can have a different surface profile than the second portion. In some embodiments, the third portion can have a similar surface profile as the first portion. The second portion can be configured to provide an enhanced vision quality metric at distance in comparison to the third portion. For example, the enhanced vision quality metric can be a modulation transfer function, a contrast sensitivity, a derivation thereof, or a combination thereof. In some embodiments, the first portion can have a shape that comprises a conic, biconic, or biaspheric envelope offset by perturbations of the envelope comprising an aspheric higher order function of radial distance from the optical axis.

Certain embodiments described herein include a lens configured for implantation into an eye of a human. The lens can include an optic comprising transparent material. The optic can have an anterior surface and a posterior surface. Each of the anterior surface and the posterior surface can have a surface vertex. The optic can have an optical axis through the surface vertices. At least one of the anterior and posterior surfaces can comprise a surface having a first portion and a second portion. The first portion can have a shape that comprises a conic, biconic, or biaspheric envelope offset by perturbations with respect to the envelope comprising an aspheric higher order function of radial distance from the optical axis. The second portion can have a shape that comprises a conic, biconic, or biaspheric envelope not offset by perturbations of the envelope comprising an aspheric higher order function of radial distance from the optical axis.

In various embodiments of the lens, the first portion can be disposed centrally about the optical axis. The second portion can surround said first portion. In some embodiments, the lens can include a third portion surrounding the second portion. The third portion can have a shape that comprises a conic, biconic, or biaspheric envelope offset by perturbations with respect to the envelope comprising an aspheric higher order function of radial distance from the optical axis. In some such embodiments, the third portion can have substantially the same conic, biconic, or biaspheric envelope offset by perturbations with respect to the envelope comprising an aspheric higher order function of radial distance from the optical axis as the first portion.

Certain embodiments described herein include a lens configured for implantation into an eye of a human. The lens can include an optic comprising transparent material. The optic can have an anterior surface and a posterior surface. Each of the anterior surface and the posterior surface can have a surface vertex. The optic can have an optical axis through the surface vertices. At least one of the anterior and posterior surfaces can comprise a surface having a first portion and a second portion. The first portion can be disposed centrally about the optical axis. The second portion can surround the first portion. The first portion can have higher spherical aberration control that provides extended depth of field than the second portion.

In various embodiments, the lens can include a third portion surrounding the second portion. The third portion can have higher spherical aberration control that provides extended depth of field than the second portion. The third portion can have substantially the same spherical aberration control as the first portion. The first portion can have a shape that comprises a conic, biconic, or biaspheric envelope offset by perturbations from the envelope comprising an aspheric higher order function of radial distance from the optical axis.

In various embodiments of the lens having a third portion, the third portion can have a shape that comprises a conic, biconic, or biaspheric envelope offset by perturbations from the envelope comprising an aspheric higher order function of radial distance from the optical axis.

In various embodiments of the lens having a shape that comprises a conic, biconic, or biaspheric envelope offset by perturbations from the envelope comprising an aspheric higher order function of radial distance from the optical axis, the aspheric higher order function can include at least one even order term, a₂ₙr²ⁿ, where n is an integer and a₂ₙ is a coefficient and r is the radial distance from the optical axis. For example, the aspheric higher order function can include a second order term, a₂r², where a₂ is a coefficient and r is the radial distance from the optical axis. As another example, the aspheric higher order function can include a fourth order term, a₄r⁴, where a₄ is a coefficient and r is the radial distance from the optical axis. The aspheric higher order function can also include a sixth order term, a₆r⁶ where a₆ is a coefficient and r is the radial distance from the optical axis. Further, the aspheric higher order function can include an eighth order term, a₈r⁸ where a₈ is a coefficient and r is the radial distance from the optical axis.

In various embodiments of the lens having a first and second portion, the lens can further comprise a transition portion providing a smooth transition without discontinuity between the first and second portions. The transition portion can have a distance between inner and outer radii in the range of about 0.1-1 mm. The first portion can have a maximum cross-sectional diameter in the range of about 2.5-4.5 mm. For example, the first portion can have a maximum cross-sectional diameter of about 3.75 mm. The second portion can have a distance between inner and outer radii in the range of about 1-3.5 mm. In some embodiments, the second portion can have a distance between inner and outer radii in the range of about 0.25-1.5 mm.

In various embodiments of the lens, the optic can have a thickness along the optical axis that is in the range of about 100-700 microns (or any range formed by any of the values in this range). Alternatively, the optic can have a thickness along the optical axis that is in the range of about 700 microns to 4 millimeters (or any range formed by any of the values in this range). In various embodiments, the lens can also include at least one haptic disposed with respect to the optic to affix the optic in the eye when implanted therein. In some embodiments, the anterior surface can comprise the surface having the first and second portions. The posterior surface can comprise a shape having a biconic envelope.

Certain embodiments described herein include a lens configured for implantation into an eye of a human. The lens can include an optic comprising transparent material. The optic can have an anterior surface and a posterior surface. Each of the anterior surface and the posterior surface can have a surface vertex. The optic can have an optical axis through the surface vertices. At least one of the anterior and posterior surfaces can comprise a surface having a first portion and a second portion. The first portion can be disposed centrally about the optical axis. The second portion can surround the first portion. The first portion can be configured to provide an extended depth of field. The second portion can be configured to provide a monofocal distance focusing.

In some such embodiments, the lens can further comprise a third portion surrounding the second portion. The third portion can be configured to provide an extended depth of field. The first portion can have a shape that comprises a conic, biconic, or biaspheric envelope offset by perturbations with respect to the envelope comprising an aspheric higher order function of radial distance from the optical axis. In addition, the third portion can have a shape that comprises a conic, biconic, or biaspheric envelope offset by perturbations with respect to the envelope comprising an aspheric higher order function of radial distance from the optical axis.

In various embodiments of the lens having first and second portions, each of the first and second portions can have a caustic. The second portion can have a conic constant such that the caustic of the second portion blends smoothly with the caustic of the first portion. In some examples, the caustic of the second portion blends more smoothly with the caustic of the first portion than if the second portion comprises a spherical surface. In various embodiments of the lens having a third portion, the second and third portions can have a caustic. The second portion can have a conic constant such that the caustic of the second portion blends smoothly with the caustic of the third portion. In some examples, the caustic of the second portion blends more smoothly with the caustic of the third portion than if the second portion comprises a spherical surface.

In certain embodiments of the lens having first and second portions, the anterior surface can be convex. The posterior surface can be concave. For example, the anterior surface can be convex and the posterior surface can be concave such that the optic is meniscus shaped. In various other embodiments, the posterior surface can be convex. In some embodiments, the anterior surface can be concave. In addition, in various embodiments of the lens having first and second portions, the second portion can have a shape that comprises a conic, biconic, or biaspheric envelope not offset by perturbations of the envelope comprising an aspheric higher order function of radial distance from the optical axis.

Certain embodiments described herein include a lens configured for implantation into an eye of a human. The lens can include an optic comprising transparent material. The optic can have an anterior surface and a posterior surface. Each of the anterior surface and the posterior surface can have a surface vertex. The optic can have an optical axis through the surface vertices. The lens can include at least one haptic disposed with respect to the optic to affix the optic in the eye when implanted therein. The anterior and posterior surfaces can comprise aspheric surfaces and the posterior surface can have an aspheric shape that comprises a biconic offset by perturbations comprising an aspheric higher order function of radial distance from the optical axis. The posterior surface can have an absolute value of ratio Rₓ/R_{y} between 0, 0.1, 0.2, 0.25, or 0.5 and 100 and an absolute value of ratio kₓ/k_{y} between 0, 0.1, 0.2, 0.25, or 0.5 and 100. In some embodiments, the absolute value of the ratio Rₓ/R_{y} is between 0, 0.1, 0.2, 0.25, or 0.5 and 75; 0, 0.1, 0.2, 0.25, or 0.5 and 50; 0, 0.1, 0.2, 0.25, or 0.5 and 25; or 0, 0.1, 0.2, 0.25, or 0.5 and 10. In addition, in some embodiments, the absolute value of the ratio kₓ/k_{y} is between 0, 0.1, 0.2, 0.25, or 0.5 and 75; 0, 0.1, 0.2, 0.25, or 0.5 and 50; 0, 0.1, 0.2, 0.25, or 0.5 and 25; or 0, 0.1, 0.2, 0.25, or 0.5 and 10.

Certain embodiments described herein include a lens configured for implantation into an eye of a human. The lens can include an optic comprising transparent material. The optic can have an anterior surface and a posterior surface. The anterior surface or posterior surface can comprise an aspheric surface. The anterior and posterior surfaces can be shaped to provide a Salvador Image Quality (SIQ) metric that is at least 0.6, 0.7, 0.8, 0.9, or 1 for at least 90%, 95%, or 98% of the object vergences within the range of 0 to +1.5 D, 0 to +2.0 D, or 0 to +2.5 D when the optic is inserted into the human eye having an aperture size of 4 to 6 millimeters. For example, the aperture size can be 6 mm.

Certain embodiments described herein include a lens configured for implantation into an eye of a human. The lens can include an optic comprising transparent material. The optic can have an anterior surface and a posterior surface. The anterior surface or posterior surface can comprise an aspheric surface. The anterior and posterior surfaces can be shaped to provide an above average psychophysical grade for at least 90%, 95%, or 98% of the object vergences within the range of 0 to +1.5 D, 0 to +2.0 D, or 0 to +2.5 D when the optic is inserted into the human eye having an aperture size of 4 to 6 millimeters or into a model eye having an aperture size of 4 to 6 millimeters. In some such embodiment, each of the anterior surface and the posterior surface can have a surface vertex. The optic can have an optical axis through the surface vertices. The anterior or posterior surface can have an aspheric shape that comprises a biconic offset by perturbations comprising an aspheric higher order function of radial distance from the optical axis.

In various embodiments, the optic can comprise an exit pupil, and the anterior and posterior surfaces can be shaped to provide a radial power profile characterized by Φ(r)=a+br²+cr⁴+dr⁶+er⁸ for wavefront at the exit pupil of the optic for an object vergence of 0 to 2.5 D where r is the radial distance from the optical axis and a, b, c, d, and e are coefficients. In some embodiments, a thickness along the optical axis can be between about 100-700 micrometers. The anterior surface can be convex and the posterior surface can be concave such that the optic is meniscus shaped.

### BRIEF DESCRIPTION OF THE DRAWINGS

FIG. 1 is a schematic illustration of the human eye.

FIG. 2 is an example lens according to certain embodiments described herein.

FIG. 3A is an ultrasound of an example lens 200 in accordance with certain embodiments described herein implanted in the eye.

FIG. 3B is the cross sectional side view of the example lens shown in FIG. 2.

FIG. 4 is a schematic of the cross sectional side view of the optic of the lens shown in FIG. 2.

FIG. 5A is a schematic of an example positive meniscus optic.

FIG. 5B is a schematic of an example negative meniscus optic.

FIG. 6A schematically illustrates the depth of field in object space and the depth of focus in image space.

FIG. 6B schematically illustrates image caustic and circle of confusion.

FIG. 6C schematically illustrates the defocus curves for a standard spherical lens and an idealized hyperfocal eye.

FIG. 6D schematically illustrates an example model to evaluate and design a lens in accordance with certain embodiments described herein.

FIGS. 7A-7B are schematics for an example anterior surface and/or a posterior surface of an optic having a first portion configured to provide extended depth of field, and a second portion configured to provide enhanced distance visual acuity.

FIGS. 8A-8B are schematics for another example anterior surface and/or a posterior surface of an optic having a first portion configured to provide extended depth of field, and a second portion configured to provide enhanced distance visual acuity.

FIG. 9A schematically illustrates an example lens inserted in the eye between the iris and the capsular bag.

FIG. 9B schematically illustrates an example of two artificial lenses inserted in the eye, a first lens and a second lens, the second artificial lens is in the capsular bag, and the first artificial lens is anterior to or forward the second lens (e.g., closer to the cornea than the second lens).

FIG. 10 is a flow diagram schematically illustrating an example method of implanting a lens into the eye.

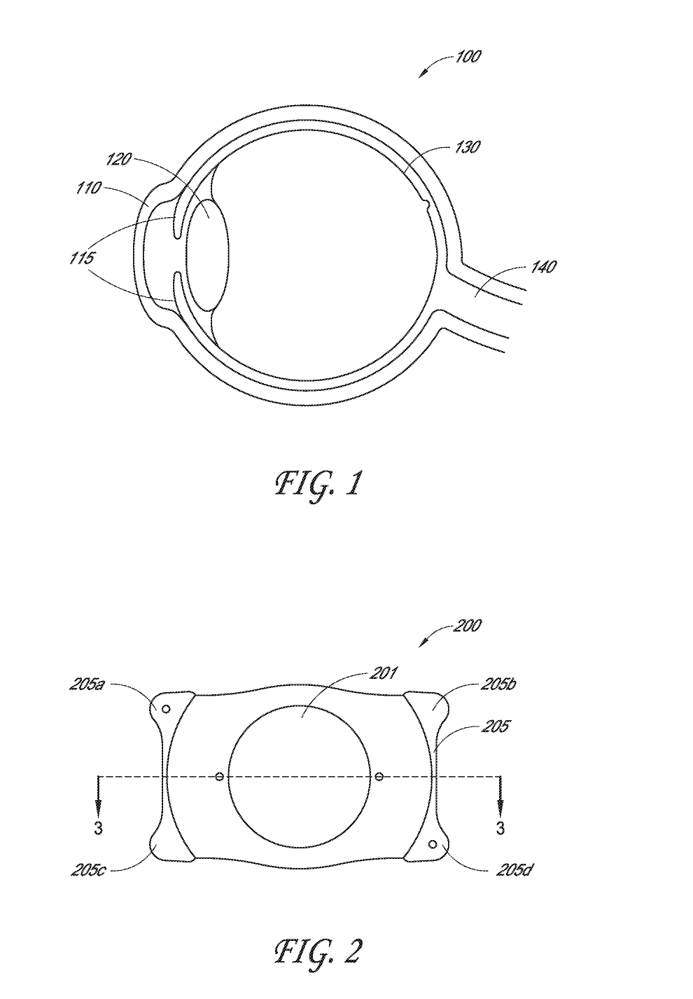

### DETAILED DESCRIPTION

Vision problems, such as myopia (nearsightedness), hyperopia (farsightedness), and astigmatism, have been corrected using eyeglasses and contact lenses. Surgical techniques, e.g., laser assisted in-situ keratomileusis (LASIK), have become more common to help address the inconvenience of eyeglasses and contact lenses. In LASIK, a laser is used to cut a flap in the cornea to access the underlying tissue, and to alter the shape of the cornea. In addition, an intraocular lens (IOL) has been used to help treat myopia and cataracts (clouding of the natural crystalline lens of the eye) by replacing the natural lens of with a pseudophakic lens configured to be secured within the capsular bag.

Another solution to treat imperfections in visual acuity is with phakic IOLs. Phakic IOLs are transparent lenses implanted within the eye without the removal of the natural crystalline lens. Accordingly, the phakic IOL together with the cornea and the crystalline lens provide optical power for imaging an object onto the retina. (In contrast, pseudophakic IOLs, which are lenses implanted within the eye to replace the natural lens, e.g., after removal of the cloudy natural lens to treat cataracts as described above.) Implantation of a phakic IOL can be employed to correct for myopia, hyperopia, as well as astigmatism, freeing a patient from the inconvenience of eyewear and contacts. Phakic IOL can also be removed, bringing the optics of the eye back toward a natural condition, or replaced to address changing vision correction or enhancement needs of the eye.

With age, people develop presbyopia (inability to focus on near objects), which has been addressed with reading glasses in order to provide the extra refractive power lost when accommodation for near objects is no longer attainable. Multifocal contact lenses and IOLs, which provide discrete foci for near and far vision, have also been used, but the losses in contrast sensitivity and the presence of coaxial ghost images in the patient's field of view have made the acceptance of such solutions limited.

Certain embodiments described herein can advantageously provide ophthalmic implants for vision correction of, including but not limited to, myopia, hyperopia, astigmatism, cataracts, and/or presbyopia with extended depth of field and enhanced visual acuity. In various embodiments, the ophthalmic implants include a lens configured for implantation into an eye of a patient, for example, a human being. Such lenses are particularly useful for treating presbyopia and onset of presbyopia in middle age populations.

Certain embodiments can include phakic lens implants, where the lens can be implanted in front of the natural crystalline lens 120, such as between the cornea 110 and the iris 115. Other embodiments are configured to be placed between the iris 115 and natural crystalline lens 120. Some example embodiments include lenses for treating myopia, hyperopia, astigmatism, and/or presbyopia.

Some other embodiments can include a pseudophakic lens implanted within the eye, for example, in the capsular bag, after removal of the crystalline lens 120. As discussed above, a pseudophakic lens can be used for treating cataracts as well as for providing refractive correction.

FIG. 2 is an example lens 200 according to various embodiments described herein. The lens 200 can include an optical zone or optic 201. The optic 201 transmits and focuses, e.g., refracts, light received by the lens 200. As will be described in more detail herein, the optic 201 can comprise a surface shape of one or more surfaces of the optic 201 designed to refract and focus light and increase the depth of field and visual acuity. For example, in some embodiments, the surface shapes of the surfaces of the optic 201 can be designed such that the optic 201 can continuously focus light for high visual acuity, e.g., 20/20 vision, for a wide range of object vergences (e.g., vergences within the range of at least about 0 to about 2.5 Diopter, in some implementations from at least about 0 diopter to at least about 1.8, 1.9, 2.0, 2.1, 2.2, 2.3, 2.4, 2.5, 2.6, 2.7, 2.8, 2.9, or 3.0 diopters or possibly from at least about 0.1, 0.2, 0.3, 0.4, 0.5, 0.6, or 0.7 diopter to at least about 2.5 2.6, 2.7, 2.8, 2.9, or 3.0 diopters) onto the retina to increase the depth of field. Furthermore, in some embodiments, the surface shapes of the surfaces of the optic 201 can be designed such that the images are substantially coaxial and of substantially similar magnitude to reduce the presence of ghost images.

As shown in FIG. 2, the example lens 200 can also include a haptic 205. In some embodiments, the haptic 205 can include one or more haptics or haptic portions 205a, 205b, 205c, and 205d to stabilize the lens in and attach the lens 200 to the eye. For example, in FIG. 2, the haptic portions 205a, 205b, 205c, and 205d are disposed about the optic 201 to affix the optic 201 in the eye when implanted therein. In certain embodiments the haptic portions 205a, 205b, 205c, and 205d are configured to stabilize the optic 201 in the eye such that the optical axis of the optic 201 is disposed along a central optical axis of the eye. In such embodiments, the stability of the wavefront of the optic 201 in the eye can be provided by the haptic portions 205a, 205b, 205c, and 205d. In various embodiments, the lens and in particular the haptics are configured to be implanted outside the capsulary bag, for example, forward the natural lens as for a phakic IOL design. As discussed above, the phakic IOL implant may be configured for implantation between the iris and the natural lens. Accordingly, in certain embodiments, the haptic 205 is vaulted such that the optic 201 is disposed along a central optical axis of the eye at a location anterior of the location of contact points between the haptic portions 205a-205d. The configuration enhances clearance between the optic 201 and the natural lens in a phakic eye, which natural lens flexes when the eye accommodates. In some cases, the haptic 205 is configured to provide minimum clearance to the natural lens when implanted that reduce, minimize or prevents contact between an anterior surface of the natural lens and a posterior surface of the optic 201. With some materials, contact between the optic 201 and the anterior surface of the natural lens is permitted. In some embodiments, the lens 200 can be implanted across the pupil or the opening of the iris 115, and when in place, the haptic portions 205a, 205b, 205c, and 205d can be placed under the iris 115. Although the haptic 205 shown in FIG. 2 includes four haptic portions 205a, 205b, 205c, and 205d in the shape of extended corner portions, the shape, size, and number of haptics or haptic portions are not particularly limited.

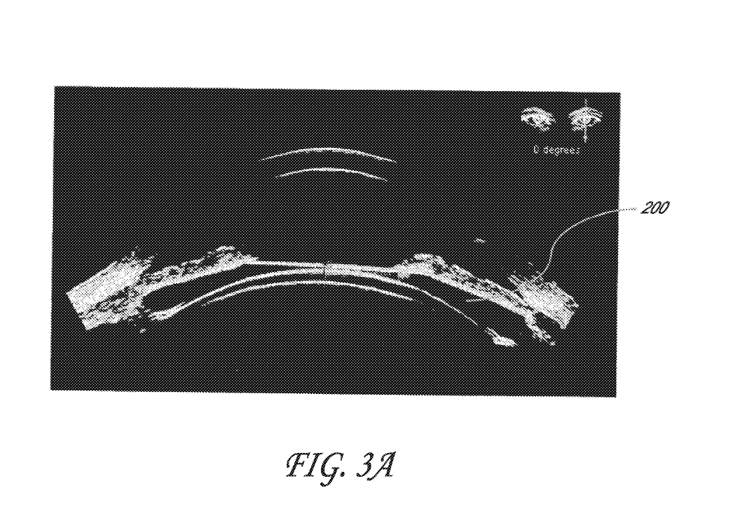

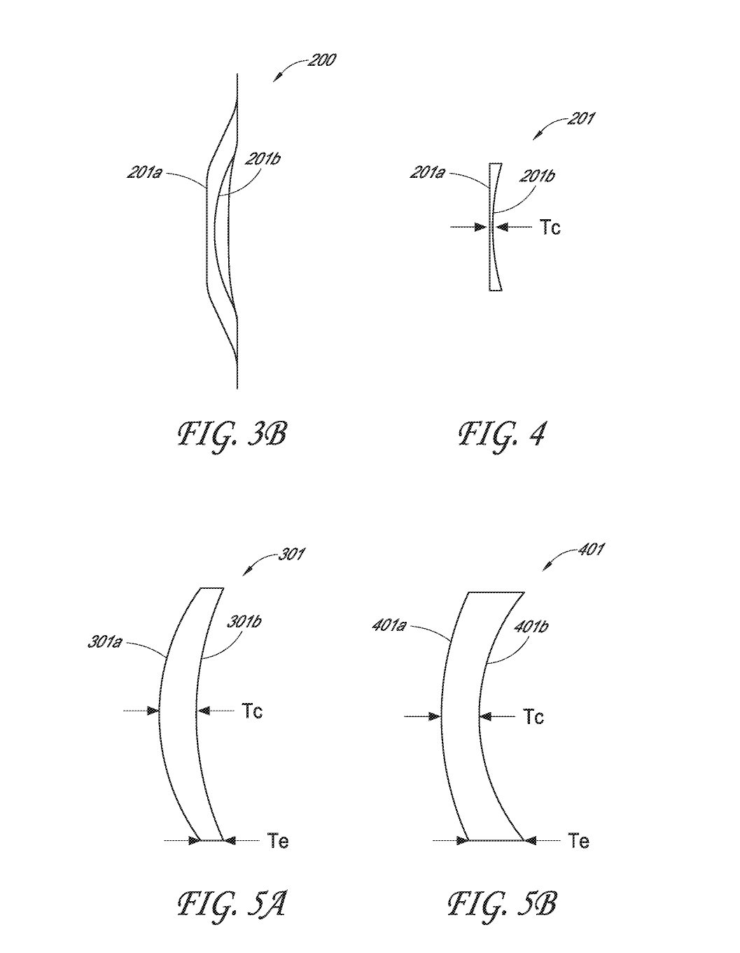

In various implementations, for example, the lens is configured for implantation within the capsular bag after removal of the natural lens. Such pseudophakic lens may have haptics having a shape, size and/or number suitable for providing secure placement and orientation within the capsular bag after implantation. FIG. 3A is an ultrasound of an example lens 200 in accordance with certain embodiments described herein implanted in the eye.

The optic 201 can include a transparent material. For example, the transparent material can include a collagen copolymer material, a hydrogel, a silicone, and/or an acrylic. In some embodiments, the transparent material can include a hydrophobic material. In other embodiments, the transparent material can include a hydrophilic material. Other materials known or yet to be developed can be used for the optic 201.

Certain embodiments of the optic 201 can advantageously include a collagen copolymer material, e.g., similar to material used in Collamer® IOLs by STAAR® Surgical Company in Monrovia, Calif. An example collagen copolymer material is hydroxyethyl methacrylate (HEMA)/porcine-collagen based biocompatible polymer material. Since collagen copolymer materials can have characteristics similar to that of the human crystalline lens, certain embodiments of the lens described herein can perform optically similar to the natural lens. For example, in some embodiments, due to the anti-reflective properties and water content of about 40%, a lens 200 made with a collagen copolymer material can transmit light similar to the natural human crystalline lens. Less light can be reflected within the eye, leading to sharper, clearer vision, and fewer occurrences of glare, halos, or poor night vision compared with lenses made with other lens materials.

In some embodiments of the lens 200 made with a collagen copolymer material, the lens 200 can be flexible, allowing easy implantation within the eye. In addition, because collagen copolymer materials are made with collagen, various embodiments of the lens 200 are biocompatible with the eye. In some embodiments, the lens 200 can attract fibronectin, a substance found naturally in the eye. A layer of fibronectin can form around the lens 200, inhibiting white cell adhesion to the lens 200. The coating of fibronectin can help prevent the lens 200 from being identified as a foreign object. In addition, like the collagen it contains, various embodiments of the lens 200 can carry a slight negative charge. Since proteins in the eye also carry a negative charge, as these two negative forces meet along the border of the lens 200, the charge repulsion can help push away the proteins from the lens 200. As such, the lens 200 can naturally keep itself clean and clear.

Furthermore, in some embodiments, the lens 200 can include an ultraviolet (UV) blocker. Such a blocker can help prevent harmful UVA and UVB rays from entering the eye. Accordingly, certain embodiments can help prevent the development of UV related eye disorders.

In some embodiments, the haptic 205 (or one or more of the haptic portions 205a, 205b, 205c, and 205d) can also be made of the same material as the optic 201. For example, the haptic 205 can be made of a collagen copolymer, a hydrogel, a silicone, and/or an acrylic. In some embodiments, the haptic 205 can include a hydrophobic material. In other embodiments, the haptic 205 can include a hydrophilic material. Other materials known or yet to be developed can also be used for the haptic 205.

The lens 200 can be manufactured by diamond turning, molding, or other techniques known in the art or yet to be developed. In some embodiments of the lens 200 manufactured with a collagen copolymer material, the lens 200 can be machined in a dry state, followed by hydration to stabilize the lens 200. A similar approach can be employed for other material as well.

FIG. 3B is the cross sectional side view of the example lens 200 shown in FIG. 2; and FIG. 4 is a schematic of the cross sectional side view of the optic 201 of the lens 200. The optic 201 has an anterior surface 201a and a posterior surface 201b. The optic 201 also has a center through which the optical axis of the lens passes and a thickness T_{c} at the center along the optical axis. The optical axis passes through the surface vertices of the anterior and posterior surfaces 201a, 201b. The exact size of the optic 201 can depend on the patient's pupil size, the material of the lens 200, and the patient's prescription. In some embodiments, for example, for phakic lenses, the thickness at the center T_{c} of the optic 201 can be made relatively thin. For example, the thickness at the center T_{c} of the optic 201 can be about 100 to about 700 micrometers, about 100 to about 600 micrometers, about 100 to about 500 micrometers, about 100 to about 400 micrometers, about 100 to about 300 micrometers, or about 100 to about 200 micrometers, such that the lens 200 can be relatively unnoticeable to the patient and to others. Thinner lenses also simplify the process of insertion of the lens through the eye tissue, e.g., cornea. For example, the optic could have a thickness along the optical axis of about 110, 115, 120, 130, 140, or 150 to about 200, 300, or 400 micrometers, any values between any of these thicknesses, or any ranges formed by any of these thicknesses. The thickness at the center T_{c} of the optic 201 can thus be any thickness in between the above mentioned values, e.g., thickness in ranges between any of the following: 100 micrometers, 110 micrometers, 115 micrometers, 120 micrometers, 130 micrometers, 140 micrometers, 150 micrometers, 200 micrometers, 250 micrometers, 300 micrometers, 350 micrometers, 400 micrometers, 450 micrometers, 500 micrometers, 550 micrometers, 600 micrometers, 650 micrometers, or 700 micrometers.

In some other embodiments for example, for pseudophakic lenses where the lens 201 replaces the natural crystalline lens, the thickness at the center T_{c} of the optic 201 can be thicker than those for phakic lenses, e.g., about 700 micrometers to about 4 mm, about 700 micrometers to about 3 mm, about 700 micrometers to about 2 mm, about 700 micrometers to about 1 mm, any value in between such ranges, or any ranges formed by any of the values in these ranges. For example, the thickness at the center T_{c} of the optic 201 can be about 700 micrometers, about 800 micrometers, about 900 micrometers, about 1 millimeter, about 1.5 millimeters, about 2 millimeters, about 2.5 millimeters, about 3 millimeters, about 3.5 millimeters, or about 4 millimeters or ranges therebetween. However, even for pseudophakic lenses the lens may employ smaller thicknesses, T_{c}, for example, thicknesses between about 300 micrometers to 700 micrometers, for example, 300 micrometers, 400 micrometers, 500 micrometers, 600 micrometers or 700 micrometers or any ranges therebetween such as 300 to 400 micrometer, 400 to 500 micrometers, 500 to 600 micrometers.

In accordance with certain embodiments described herein, the anterior surface 201a is convex and the posterior surface 201b is concave such that the optic 201 is meniscus shaped. FIGS. 5A and 5B are example cross sectional side views of the optic 201 being meniscus shaped. A meniscus shaped optic 201 can be quite advantageous when used for example, in a phakic lens. For example, when implanted behind (or posterior of) the iris and in front of (or anterior of) the natural lens, an anterior surface 201a of the optic 201 that is convex can help prevent chaffing of the iris adjacent to that surface 201a, and a posterior surface 201b of the optic 201a that is concave can help prevent damage to the natural lens adjacent to that surface 201b, which may result in, for example, cataracts.

The meniscus shaped optic can be described as either positive or negative. As shown in FIG. 5A, a positive meniscus optic 301 has a steeper curving convex surface 301a than the concave surface 301b, and has a greater thickness at the center T_{c} (through which the optical axis passes) than at the edge Tₑ. In contrast, as shown in FIG. 5B, a negative meniscus optic 401 has a steeper curving concave surface 401b than the convex surface 401a, and has a greater thickness at the edge Tₑ than at the center T_{c}. In certain embodiments, a positive meniscus optic can be used to treat hyperopia, while in other embodiments, a negative meniscus optic can be used to treat myopia.

In various embodiments, the optic 201 is not meniscus shaped. For example, in some embodiments, the anterior surface 201a is substantially flat and the posterior surface 201b is concave such that the optic 201 is plano-concave. In other embodiments, both the anterior surface 201a and the posterior surface 201b are concave such that the optic 201 is biconcave. In further embodiments, the anterior surface 201a is convex and the posterior surface 201b is substantially flat such that the optic 201 is plano-convex. In yet further embodiments, both the anterior surface 201a and the posterior surface 201b are convex such that the optic 201 is biconvex.

In certain embodiments, the anterior surface 201a and/or the posterior surface 201b of the optic 201 can include aspheric surfaces. For example, the anterior surface 201a and/or the posterior surface 201b of the optic 201 can include a surface shape that is not a portion of a sphere. In various embodiments, the anterior surface 201a and/or the posterior surface 201b can be rotationally symmetric. For example, the surface profile or sag of the aspheric shape can include at least a conic term. The conic term can be described as:

$$z = \frac{{cr}^{2}}{1 + \sqrt{1 - ( 1 + k ) {c}^{2} {r}^{2}}} , \qquad ( 1 )$$

where c is the curvature of the surface (or the inverse of the radius), k is the conic constant, and r is the radial distance from the surface vertex.

In some embodiments, the aspheric shape can include a conic offset by perturbations comprising, for example, a higher order function of radial distance from the surface vertex. Thus, the sag of the aspheric shape can include the conic term and a higher order function of radial distance from the surface vertex. The higher order function can describe the aspheric perturbations from the conic term. In some embodiments, the higher order function can include at least one even order term a₂ₙr²ⁿ, where n is an integer, a₂ₙ is a coefficient, and r is the radial distance from the surface vertex. For example, the aspheric shape can be described using the conic term and the even-powered polynomial terms (e.g., describing an even asphere):

$$z ( r ) = \frac{{cr}^{2}}{1 + \sqrt{1 - ( 1 + k ) {c}^{2} {r}^{2}}} + {a}_{2} {r}^{2} + {a}_{4} {r}^{4} + {a}_{6} {r}^{6} + {a}_{8} {r}^{8} + … . \qquad ( 2 )$$

As can be seen in the example equation (2), the higher order function can include at least a second order term (a₂r²), a fourth order term (a₄r⁴), a sixth order term, (a₆r⁶), and/or an eighth order term (a₈r⁸). In some embodiments, the higher order function can include one or more odd order terms. For example, the higher order function can include only odd order terms or a combination of even and odd order terms.

As also shown in equation (2), the surface shape can depend on the conic constant k. If the conic constant k=0, then the surface is spherical. Thus, in some embodiments, k has a magnitude of at least zero, such that |k|≥0. In some embodiments, k has a magnitude greater than zero, such that |k|>0. In various embodiments, k has a magnitude of at least one, such that |k|≥1. In some embodiments, |k|≥2, |k|≥3, |k|≥5, |k|≥7, or |k|≥10. For example, k≤−1, k≤−2, k≤−3, k≤−5, k≤−7, k≤−10. In various embodiments, therefore, the surface has a shape of a hyperbola. However, in certain embodiment, the magnitude of the conic constant may be less than one, e.g., 0≤|k|≤1.

In various embodiments, the anterior surface 201a and/or the posterior surface 201b can be rotationally non-symmetric and have different curvature along different directions through the center and/or optical axis of the optic 201. For example, the anterior surface 201a and/or the posterior surface 201b can have different curvature along orthogonal directions through the center of the optic 201. Certain such embodiments can be advantageous for treating astigmatism, where correction along different directions (meridians) can be desired.

In some embodiments, the sag of the rotationally non-symmetric surface can include at least a biconic term. A biconic surface can be similar to a toroidal surface with the conic constant k and radius different in the x and y directions. The biconic term can be described as:

$$z = \frac{{c}_{x} {x}^{2} + {c}_{y} {y}^{2}}{1 + \sqrt{1 - ( 1 + {k}_{x} ) {c}_{x}^{2} {x}^{2} - ( 1 + {k}_{y} ) {c}_{y}^{2} {y}^{2}}} , \qquad ( 3 )$$

where cₓ is the curvature of the surface in the x direction (or the inverse of the radius in the x direction), and c_{y} is the curvature of the surface in the y direction (or the inverse of the radius in the y direction) while kₓ is the conic constant for the x direction, and k_{y} is the conic constant for the y direction.

In some embodiments, the aspheric shape can include the biconic offset by perturbations comprising a higher order function of radial distance from the surface vertex. Thus, similar to equation (2), the sag of the aspheric shape can include the biconic term and a higher order function. The higher order function can include at least one even order term, e.g., at least a second order term (a₂r²), a fourth order term (a₄r⁴), a sixth order term, (a₆r⁶), and/or an eighth order term (a₈r⁸). For example, similar to equation (2), the higher order function can be a₂r²+a₄r⁴+a₆r⁶+a₈r⁸+ . . . .

In some embodiments, the higher order function can include one or more odd order terms. For example, the higher order function can include only odd order terms or a combination of even and odd order terms.

Accordingly, as described herein, the anterior surface 201a and/or the posterior surface 201b of the optic 201 can have a shape that includes a conic term (with or without a higher order function) or a biconic term (with or without a higher order function).

One example for vision correction for presbyopia and/or astigmatism includes an anterior surface 201a and a posterior surface 201b both having an aspheric surface. The aspheric surface of the anterior surface 201a has a shape that includes a conic term offset by perturbations comprising second, fourth, sixth, and eighth order terms; and the aspheric surface of the posterior surface 201b has a shape that includes a biconic term. The sag of the example aspheric anterior surface 201a can be given as:

$$z ( r ) = \frac{{cr}^{2}}{1 + \sqrt{1 - ( 1 + k ) {c}^{2} {r}^{2}}} + {a}_{2} {r}^{2} + {a}_{4} {r}^{4} + {a}_{6} {r}^{6} + {a}_{8} {r}^{8} . \qquad ( 4 )$$

Furthermore, the sag of the example posterior surface 201b, which can be biconic, can be given as:

$$z = \frac{{c}_{x} {x}^{2} + {c}_{y} {y}^{2}}{1 + \sqrt{1 - ( 1 + {k}_{x} ) {c}_{x}^{2} {x}^{2} - ( 1 + {k}_{y} ) {c}_{y}^{2} {y}^{2}}} , \qquad ( 5 )$$

which is similar to equation (3). Certain embodiments of such a lens may be, although is not limited to, a meniscus lens.

Other examples are possible. In certain embodiments, the particular shape (e.g., curvature of anterior surface, curvature of posterior surface, conic constants, coefficients of the higher order function, etc.) of the optic 201 can depend on the patient's prescription.

As some examples, for lenses having a nominal dioptric power between about −18 D to about 6 D sphere with 0 to about 2 D cylinder, with 0 to about 3 D cylinder, or with 0 to about 4 D cylinder, the following non-limiting example design parameters can be used in certain embodiments. The radius R of the anterior surface (e.g., the inverse of the curvature) can be between about −100 mm to about 100 mm, about −50 mm to about 50 mm, about −10 mm to about 10 mm, or about −5 mm to about 5 mm. In some examples, R of the anterior surface can be between about −1 mm to about 1 mm or 0 to about 1 mm. For example, the radius of the anterior surface can be between 0 to about 1×10⁻² mm, between about 1×10⁻⁷ mm to about 5×10⁻³ mm, between about 1×10⁻⁶ mm to about 1×10⁻³ mm, or between about 5×10⁻⁶ mm to about 5×10⁻⁴ mm.

As described herein, in various embodiments, k of the anterior surface can have a magnitude greater than zero such that |k|>0. In some embodiments, k has a magnitude of at least one, such that |k|≥1. In some embodiments, |k|≥2, |k|≥3, |k|≥5, |k|≥7, or |k|≥10. For example, k≤−1, k≤−2, k≤−3, k≤−5, k≤−7, k≤−10. In some embodiments, k<<−10. For example, in some embodiments, k can be between about −1×10⁶ to −100, between about −5×10⁵ to about −5×10⁴, or between about −3×10⁵ to about −2×10⁵.

Accordingly, in various embodiments the magnitude of the ratio of the conic constant of the anterior surface and the radius of curvature of the anterior surface may be between 10⁴ and 10¹⁴, between 10⁶ and 10¹², between 10⁸ and 10¹¹, between 10⁹ and 10¹¹, between 10⁸ and 10¹⁰, between 10⁹ and 10¹⁰ in various embodiments.

The coefficient a₂ for the second order term of the anterior surface in various embodiments can be between 0 to about 1. For example, a₂ can be between 0 to about 0.5, between about 0.001 to about 0.3, or between about 0.005 to about 0.2.

The coefficient a₄ for the fourth order term of the anterior surface in various embodiments can be between about −1 to 0. For example, a₄ can be between about −0.1 to 0, between about −0.05 to about −1×10⁻⁴, or between about −0.01 to about −1×10⁻³.

The coefficient a₆ for the sixth order term of the anterior surface in various embodiments can be between 0 to about 1. For example, a₆ can be between 0 to about 0.1, between 0 to about 0.01, or between about 0.0001 to about 0.001.

In addition, the coefficient a₈ for the eighth order term of the anterior surface in various embodiments can be between about −1 to 0. For example, a₈ can be between about −0.001 to 0, between about −0.0005 to 0, or between about −0.0001 to 0.

Furthermore, for lenses having a nominal dioptric power between about −18 D to about 6 D sphere with 0 to about 2 D cylinder, with 0 to about 3 D cylinder, or with 0 to about 4 D cylinder, the following non-limiting example design parameters can be used in certain embodiments for the posterior surface. The radius R_{y} of the posterior surface in the y direction (e.g., the inverse of the curvature in the y direction) can be between 0 to about 20 mm. For example, the radius R_{y} of the posterior surface can be between 0 to about 15 mm, between about 2 mm to about 13 mm, or between about 3 mm to about 14 mm, or between about 4 mm to about 10 mm.

In various embodiments, k_{y} of the posterior surface can be between about −20 to about 20, between about −18 to about 15, or between about −15 to about 5. In some such embodiments, k_{y} of the posterior surface does not necessarily have a magnitude of at least one. For example, k_{y} can be between about −1 to about 1. In various embodiments, |k_{y}| is greater than zero.

The radius Rₓ of the posterior surface in the x direction (e.g., the inverse of the curvature in the x direction) can be between 0 to about 20 mm. For example, the radius of the posterior surface can be between 0 to about 15 mm, between 0 to about 12 mm, or between 0 to about 10 mm.

In various embodiments, kₓ of the posterior surface can be between about −25 to 0, between about −20 to 0, between about −18 to 0, between about −17.5 to 0, or between about −15.5 to 0. In various embodiments, |kₓ| is greater than zero.

In certain embodiments described herein, for lenses having a nominal dioptric power between about −18 D to about 6 D sphere with 0, 0.1, 0.2, 0.25, or 0.5 to about 10 D cylinder, or any ranges between any combination of these values (e.g., with 0.1 to about 2 D cylinder, with 0.5 to about 2 D cylinder, with 0.1 to about 3 D cylinder, with 0.5 to about 3 D cylinder, with 0.1 to about 4 D cylinder, with 0.5 to about 4 D cylinder, with 0.1 to about 5 D cylinder, with 0.5 to about 5 D cylinder, with 0.1 to about 6 D cylinder, with 0.5 to about 6 D cylinder, with 0.1 to about 7 D cylinder, with 0.5 to about 7 D cylinder, with 0.1 to about 8 D cylinder, with 0.5 to about 8 D cylinder, with 0.1 to about 9 D cylinder, with 0.5 to about 9 D cylinder, with 0.1 to about 10 D cylinder, with 0.5 to about 10 D cylinder, or any ranges between any combination of these values), the posterior surface can have a shape that includes a biconic term (with or without a higher order function). In some such embodiments, the posterior surface can have an absolute value of ratio Rₓ/R_{y} between 0, 0.1, 0.2, 0.25, or 0.5 and 100, or any ranges between any combination of these values (e.g., between 0 and 100, between 0.1 and 100, between 0.5 and 100, between 0 and 75, between 0.1 and 75, between 0.5 and 75, between 0 and 50, between 0.1 and 50, between 0.5 and 50, between 0 and 25, between 0.1 and 25, between 0.5 and 25, between 0 and 10, between 0.1 and 10, or between 0.5 and 10, or any ranges between any combination of these values). In various embodiments, the absolute value of ratio Rₓ/R_{y} is greater than zero. In addition, in some embodiments, the posterior surface can have an absolute value of ratio kₓ/k_{y} between 0, 0.1, 0.2, 0.25, or 0.5 and 100, or any ranges between any combination of these values (e.g., between 0 and 100, between 0.1 and 100, between 0.5 and 100, between 0 and 75, between 0.1 and 75, between 0.5 and 75, between 0 and 50, between 0.1 and 50, between 0.5 and 50, between 0 and 25, between 0.1 and 25, between 0.5 and 25, between 0 and 10, between 0.1 and 10, or between 0.5 and 10, or any ranges between any combination of these values). In various embodiments, the absolute value of ratio kₓ/k_{y} is greater than zero.

In some embodiments, the shape of the posterior surface can be related to the shape of the anterior surface. In some such embodiments, the posterior surface can have a shape that includes a biconic term (with or without a higher order function); and the anterior surface can have a shape that includes a conic term (with or without a higher order function). The relationship of the anterior and posterior surfaces can be non-linear. In various embodiments, a pattern can exist between Rₓ, R_{y}, kₓ, k_{y}, of the posterior surface and the conic constant k of the anterior surface. For example, the absolute value of ratio Rₓ/R_{y} can be as described herein, the absolute value of kₓ/k_{y} can be as described herein, and the conic constant k of the anterior surface can be less than −2×10⁴, and in some cases <<−2×10⁴. For example, the conic constant k of the anterior surface can be between −9×10⁵ to −1×10⁶, between −8×10⁵ to −1×10⁶, between −7×10⁵ to −1×10⁶, between −6×10⁵ to −1×10⁶, or between −5×10⁵ to −1×10⁶.

In various embodiments described herein, the lenses may be utilized in a relatively low-to-zero spherical power configuration, with the addition of relatively significant cylindrical correction, e.g., greater than or equal to +1.0 D cylinder to the spherical base, in order to provide a given patient with better retinal image quality in cases where age-induced aberrations of the eye or cataract surgery-induced astigmatism may be negatively impacting the quality of life of the patient. For example, the low-to-zero spherical power configuration can include between 0, 0.1, 0.2, 0.25, or 0.5 to 3 D sphere, 1 to 3 D sphere, 2 to 5 D sphere, or 3 to 6 D sphere, with the addition of +1.0 D to +10 D cylinder or any ranges between these values (e.g, +1.0 D cylinder to +2.0 D cylinder, +2.0 D cylinder to +3.0 D cylinder, +3.0 D cylinder to +4.0 D cylinder, +4.0 D cylinder to +5.0 D cylinder, +5.0 D cylinder to +6 D cylinder, +6.0 D cylinder to +7.0 D cylinder, +7.0 D cylinder to +8.0 D cylinder, +8.0 D cylinder to +9.0 D cylinder, or +9.0 D cylinder to +10.0 D cylinder, or any ranges between any combination of these values) to the spherical base. In some embodiments, the ratio sphere/cylinder can be between 0, 0.1, 0.2, 0.25, or 0.5 to 6, or any ranges between any combination of these values (e.g., between 0 to 1, between 0.25 to 1, between 0 to 2, between 0.25 to 2, between 0 to 3, between 0.25 to 3, between 0 to 4, between 0.25 to 4, between 0 to 5, between 0.25 to 5, between 0 to 6, between 0.25 to 6, between 1 to 6, or between 2 to 6, or any ranges between any combination of these values).

Although the example design parameters of R, k, a₂, a₄, a₆, and a₈ for lenses having the above given nominal dioptric power were given for the anterior surface, and the example design parameters of R_{y}, k_{y}, Rₓ, kₓ, ratio Rₓ/R_{y}, and ratio kₓ/k_{y} were given for the posterior surface, the ranges of values for R, k, a₂, a₄, a₆, and a₈ can be used for the posterior surface, and the ranges of values for R_{y}, k_{y}, Rₓ, and kₓ, ratio Rₓ/R_{y}, and ratio kₓ/k_{y} can be used for the anterior surface. Additionally, although the anterior surface included the higher order aspheric perturbation terms (e.g., a₂, a₄, a₆, and a₈), the higher order aspheric perturbation terms (e.g., a₂, a₄, a₆, and a₈) can be used for the posterior surface instead of the anterior surface or for both the anterior and posterior surfaces. Any one or more of the values in these ranges can be used in any of these designs.

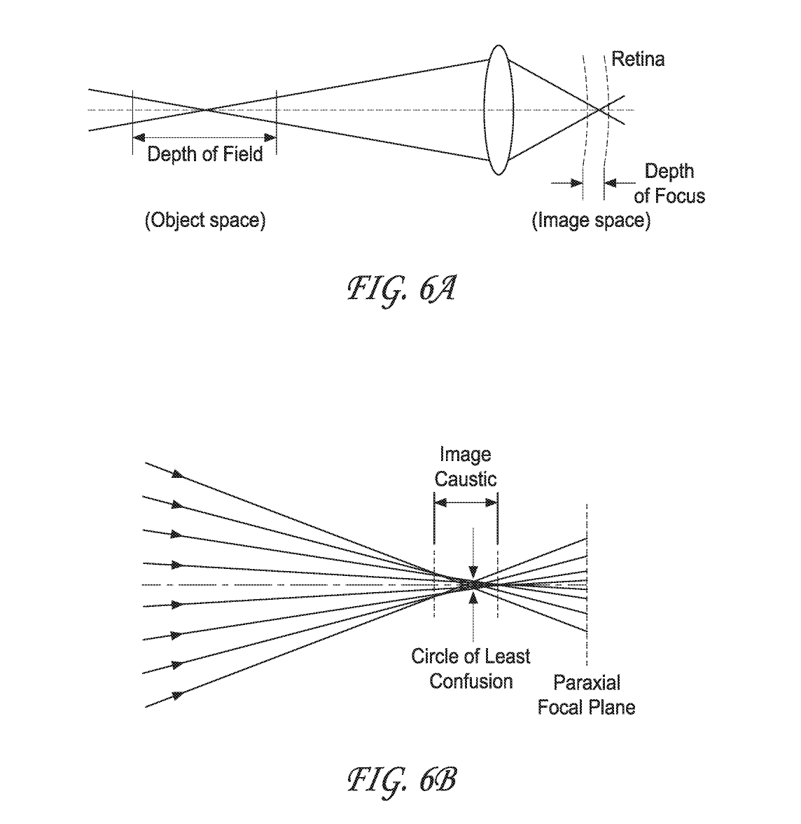

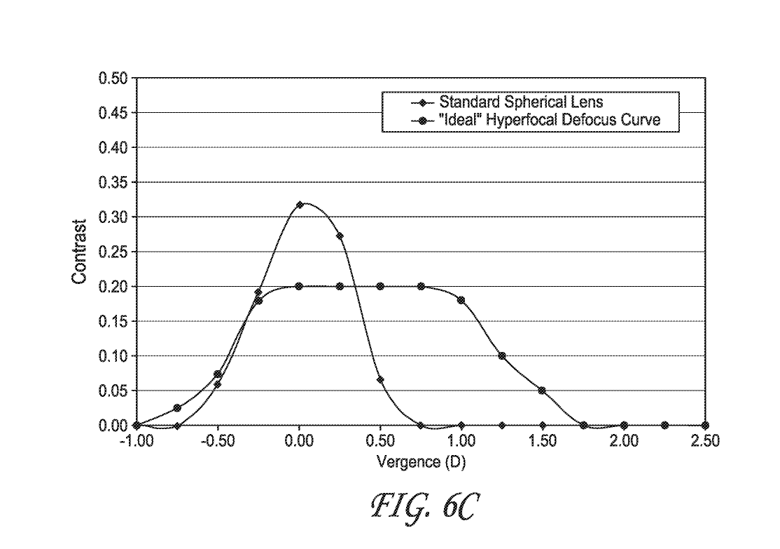

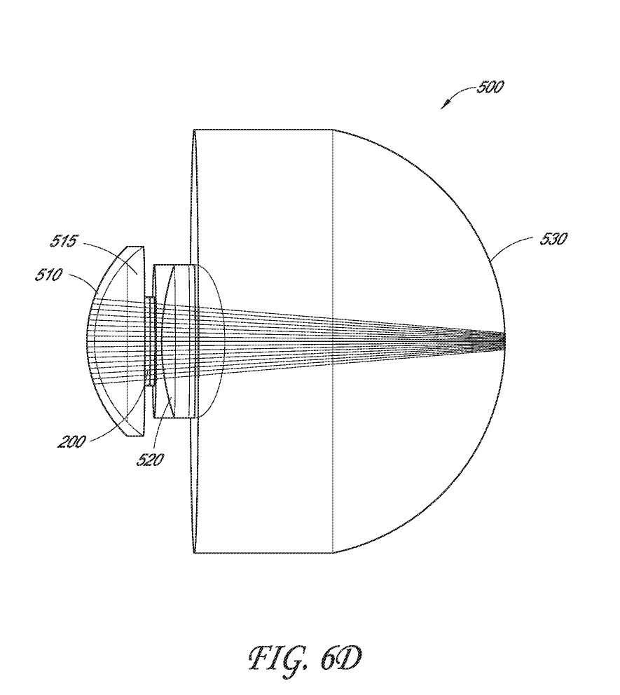

Furthermore, as described herein, the particular shape of various embodiments can be designed to increase the depth of field and to increase visual acuity. As shown in FIG. 6A, the depth of field can be described as the distance in front of and beyond the subject in object space that appears to be in focus. The depth of focus can be described as a measurement of how much distance exists behind the lens in image space wherein the image will remain in focus. To increase the depth of field, the surface shape of the anterior surface 201a and/or the surface shape of the posterior surface 201b of the optic 201 can be such that for a wide range of object vergences, the light rays are focused onto the retina or sufficiently close thereto. To increase visual acuity and reduce ghosting, the surface shape of the anterior 201a and/or the surface shape of the posterior surface 201b of the optic 201 also can be such that the images for an on-axis object are substantially on-axis and of similar magnitude with each other.

In certain such embodiments, the image caustic can be sculpted for the vergence range of about 0 to about 2.5 Diopters or more although this range may be larger or smaller. As shown in FIG. 6B, in some embodiments, the image caustic can be described as the envelop produced by a grid of light rays, and the circle of confusion can be described as an optical spot caused by a cone of light rays from a lens not coming to a perfect focus when imaging a point source. Thus, the image caustic can be sculpted such that the circle of confusion is substantially stable having a similar sizes for a range of longitudinal positions along the optical axis and relatively small. The design may sacrifice the size of the circle of confusion at some longitudinal positions along the optical axis to permit the circle of confusion to be larger for others longitudinal positions with the net result of providing circles of confusion having similar size over a range of longitudinal positions along the optical axis.

In certain embodiments, the surface shape of the anterior surface 201a and/or the surface shape of the posterior surface 201b can be determined such that the image caustic is sculpted around the hyperfocal plane of the eye. In some embodiments, the hyperfocal distance can be described as the focus distance which places the maximum allowable circle of confusion at infinity, or the focusing distance that produces the greatest depth of field. Accordingly, in certain embodiments, to increase the depth of field, the surface shape of the anterior surface 201a and/or the surface shape of the posterior surface 201b of the optic 200 can be such that the light rays are refocused to the hyperfocal distance.

In various embodiments, the surface shape of the anterior surface 201a and/or the surface shape of the posterior surface 201b of the optic 201 can be evaluated and designed using the defocus curves of the lens. A defocus curve can portray the response of a retinal image quality parameter, such as contrast, as a function of different vergences. An object at infinity has a vergence of 0 Diopter. FIG. 6C illustrates the defocus curves for a standard spherical lens and an idealized hyperfocal eye. As shown in the figure, although the contrast can decrease (due to preservation of the areas under the curves), the idealized hyperfocal eye has a stable or substantially stable (e.g., similar or substantially constant) contrast for a range of vergences.

In certain embodiments, the surface shape of the anterior surface 201a and/or the surface shape of the posterior surface 201b of the optic 201 can be evaluated and/or designed using the Liou-Brennan model eye such as under Best Corrected Distance Visual Acuity (BCDVA) conditions. FIG. 6D illustrates a schematic of an example phakic lens according to certain embodiments described herein modeled with the Liou-Brennan model eye. As shown in FIG. 6D, the lens 200 can be positioned between the iris 515 and in front of the “natural” crystalline lens 520 in the model. As also shown in FIG. 6D, the model can simulate light rays entering the eye 500 through the cornea 510, the lens 200, and the “natural” crystalline lens 520 and towards the retina 530. The model can be used for the polychromatic wavelengths between the range of about 400 nanometers to about 700 nanometers. The model can also be used with a dual-gradient index lens profile (e.g., to model astigmatism). Pseudophakic lenses according to certain embodiments described herein can also be modeled with the Liou-Brennan model eye with the lens positioned in place of the “natural” crystalline lens 520.

Other models known in the art or yet to be developed can also be used. For example, the surface shape of the anterior surface 201a and/or the surface shape of the posterior surface 201b of the optic 201 can also be evaluated and/or designed using a Badal model eye, an Arizona model eye (University of Arizona model), an Indiana model eye (Indiana University model), an ISO model eye, or any standardized or equivalent model eye. In addition, the simulations can be performed using ray tracing and/or design software known in the art or yet to be developed. As one example software, Zemax design software by Zemax, LLC in Redmond, Wash. can be used for some embodiments. The physical limitations of the environment, for example, the placement of the IOL anterior to the natural lens are useful for performing simulations for a phakic lens design. Such simulations can simultaneously evaluate performance (e.g., RMS wavefront error across the complete pupil) for multiple vergences an include contributions from the different vergences in a merit function that is optimized. Multiple wavefronts are thus evaluated in unison to arrive at a balanced design that provides substantially similar sized circles of confusion through a range of locations along the optical axis. Varying pupil size for different vergences can also be employed.

In certain embodiments, the surface shape of the anterior surface 201a and/or the surface shape of the posterior surface 201b of the optic 201 can be advantageously evaluated and designed such that for the visible wavelengths, light from an on-axis object is focused substantially on-axis, with substantially similar magnitude, and substantially on the retina within the range of at least about 0 Diopter to about 2.5 Diopter. By controlling the different orders of spherical aberrations (e.g., which can be correlated with the higher order aspheric terms in equation (2)) to achieve a substantially similar size cross-sections of the caustic for different longitudinal positions along the optical axis near the retina, and including the toric balancing and correction (e.g., the biconic term in equation (3)) when necessary to treat patients with astigmatism, the radial power profile of the lens 200 can be described as:

Φ(r)=a+br²+cr⁴+dr⁶+er⁸, (6)

where a, b, c, d, and e are real numbers. Additionally, in various embodiments, the surface shape of the anterior surface 201a and/or the surface shape of the posterior surface 201b of the optic 201 can be evaluated and designed to account for the Stiles-Crawford effect. Furthermore, the surface shapes can also be designed to consider the pupil sizes varying with illumination and/or object vergence.

In certain embodiments described herein, the design parameters (e.g., R_{y}, k_{y}, Rₓ, kₓ, ratio Rₓ/R_{y}, and ratio kₓ/k_{y} for the posterior surface and/or R, k, a₂, a₄, a₆, and a₈ for the anterior surface) can be determined for the maximum aperture for the desired toric correction. For example, the toric correction with a relatively stable caustic for a maximum aperture of 4.0 mm may be different from the toric correction with a relatively stable caustic for a maximum aperture of 3.0 mm or 5.0 mm.

To describe the performance of the lens 200, the modulation transfer function (MTF) can be used in some embodiments. For example, the MTF can describe the ability of the lens 200 to transfer contrast at a particular resolution from the object to the image. In various embodiments of the lens 200, the anterior surface 201a and the posterior surface 201b can be shaped to provide MTF values for wavelengths between the range of about 400 nanometers to about 700 nanometers (weighted by photopic, scotopic and/or mesopic distributions) that are between about 0.1 and about 0.4 at spatial frequencies of about 100 line pairs per millimeter (e.g., 20/20 vision) for at least about 90%, at least about 95%, at least about 97%, at least about 98%, or at least about 99% of the object vergences within the range of at least about 0 Diopter to about 2.0, 2.1, 2.2, 2.3, 2.4 or 2.5 Diopter (or to about 2.6, 2.7, 2.8, 2.9, 3.0) when the optic 201 is inserted into an eye. For example, the eye could be a human eye having an aperture diameter of at least about 2 millimeters, at least about 3 millimeters, at least about 4 millimeters, for example, 2 to 6 millimeters, 3 to 6 millimeters, or 4 to 6 millimeters. The MTF values may thus be 0.1, 0.2, 0.3, or 0.4 or any range therebetween. Additionally, in various implementations, the anterior and posterior surfaces are shaped to provide modulation transfer functions without phase reversal for at least 90%, 95%, or 97%, up to 98%, 99%, or 100% of the object vergences within the range of 0 D to 2.5 D (or alternatively to 2.0, 2.1, 2.2, 2.3, 2.4, 2.6, 2.7, 2.8, 2.9, or 3.0 Diopter) when said optic is inserted into a model eye having an aperture size of 2 to 6 millimeters, 3 to 6 millimeters, or 4 to 6 millimeters. In some embodiments, when the human eye includes a crystalline lens, such MTF values can be provided when the optic 201 is inserted anterior of the crystalline lens. In other embodiments, when the human eye excludes a crystalline lens, such MTF values can be provided when the optic 201 is inserted in place of the crystalline lens. The MTF values may comprise average MTF values and may be calculated by integrating over the wavelength range which is weighted by any of the photopic, scotopic, mesopic distributions or combinations thereof.

As other examples, the eye could be a model eye (e.g., Liou-Brennan, Badal, Arizona, Indiana, ISO model eye, or any standardized or equivalent model eye) that models the human eye as opposed to a human eye itself. For example, the model eye in some embodiments can also include a Liou-Brennan model eye. In some embodiments, such MTF values can be provided when the optic 201 is inserted in the model eye in a phakic configuration. In other embodiments, such MTF values can be provided when the optic 201 is inserted in a pseudophakic configuration.

Other metrics to describe the performance of the lens 200 can also be used. For example, a normalized MTF metric, such as the Salvador Image Quality (SIQ) metric, can be used. The Salvador Image Quality metric can be described as:

$$SIQ = {[ \frac{AreaUnderMTFCurve ( EyeOfInterest )}{AreaUnderMTFCurve ( StdEye )} ]}_{0 \leq ξ \leq 100 {mm}^{- 1}} \qquad ( 7 )$$

The Area Under MTF Curve can be the positive area under a given MTF curve, from zero to a spatial frequency ζ of 100 cycles/mm or the cutoff frequency, whichever appears first in the given plot. The “standard eye” can include a model eye (e.g., the Liou-Brennan model eye with a dilated, 6.0 mm diameter pupil) for the normalization. The MTF for the eye of interest can be the measured MTF of a given patient's eye, at a given wavelength (e.g., with a 6.0 mm dilated pupil). It can be measured and compared at the same angular field as the reference baseline. In the case of non-rotationally symmetric ocular systems, the results for Saggital SIQ and Tangential SIQ can be averaged. The Saggital SIQ can be calculated from the MTF in the XZ plane, whereas the Tangential SIQ can be calculated from the MTF in the YZ plane.

In various embodiments

| | |
|---|---|
| SIQ ≥ 1 | “Fighter Pilot” |
| SIQ ≈ 1 | “Emmetropic Eye” |
| SIQ < 1 | Refractive Errors Present |
| SIQ << 1 | Patient with Low Vision |

In certain embodiments described herein, the anterior and posterior surfaces can be shaped to provide a SIQ metric that is at least 0.6, 0.7, 0.8, 0.9, or 1 for at least 90%, 95%, or 97%, up to 98%, or 100% of the object vergences within the range of 0 to +1.5 D, 0 to +2.0 D, or 0 to +2.5 D when the optic is inserted into the human eye of the person whose correction is being provided having an aperture size of 4 to 6 millimeters (e.g., 4 mm, 5 mm, or 6 mm).

As another example, a psychophysical grade (e.g., standard psychophysical practices in imaging science) can be used to describe the performance of the lens. In certain embodiments described herein, the anterior and posterior surfaces can be shaped to provide an above average psychophysical grade (e.g., “good” or better) for at least 90%, 95%, or 97%, up to 98%, or 100% of the object vergences within the range of 0 to +1.5 D, 0 to +2.0 D, or 0 to +2.5 D when the optic is inserted into the human eye of the person whose correction is being provided having an aperture size of 4 to 6 millimeters (or into a model eye having an aperture size of 4 to 6 millimeters having vision similar to the person whose correction is being provided) Any grade lower than an above average psychophysical grade can determine the myopic edge for the performance of the lens. The myopic edge can be the limit of the near vision provided by the extended depth of field (e.g., +1.5 D, +2.0 D, or +2.5 D) of the lens.

Various implementations described herein comprise a single refractive lens that can be implanted in the eye, for example, posterior of the cornea. In certain implementations the refractive lens is configured to be implanted between the iris and the natural lens. In other implementations, the refractive lens is configured to be implanted in the capsular bag after removal of the natural lens. In various implementations, the refractive lens is not a diffractive lens and is devoid of a diffraction grating on the surfaces thereof. In various implementations, the refractive lens does not have discrete spaced apart foci. The anterior and posterior surfaces, for example, are shaped so as not to produce discrete foci where light is focused along the optical axis of the lens that are spaced apart from each other by regions where light is substantially less focused as provided in conventional multifocal lenses. Such multifocal design with discrete foci have multiple peaks of focused energy or of energy density at different locations on the optical axis.

Various implementations described herein can provide treatment for early onset and progression of presbyopia without need for laser surgery or reading glasses. Implementations may provide about 2.0 D of near as well as intermediate viewing. Depth of field for range over 2 D for an aperture of 5.0 mm can be provided.

Various embodiments may be employed to provide modified monovision solutions. For example, a first lens may be provided that has an extended depth of focus for object vergences over 0 to 2.0 D or over 0 to 2.5 D and second lens may be provided that has an extended depth of focus for object vergences over −2.0 to 0 D or over −2.5 to 0 D. These respective lenses may be implanted in the patient's dominant and non-dominant respectively. A patient may then be provided with extended depth's of field that are different for each of the left and right eye. However the aggregate depth of field is larger than provided by one of the first or second lenses along. The design details of such lenses may otherwise be similar to those discussed above.

As described herein, various embodiments include a lens with extended depth of field. For example, with reference to lens 200 described herein (e.g., as shown in FIGS. 2-4), the lens 200 can include an optic 201 having an anterior surface 201a and/or a posterior surface 201b having a shape designed to increase the depth of field. In certain embodiments, the anterior surface and/or the posterior surface of the optic can also include a portion designed to improve distance vision (e.g. enhance distance visual acuity) yet still provide extended depth of field.

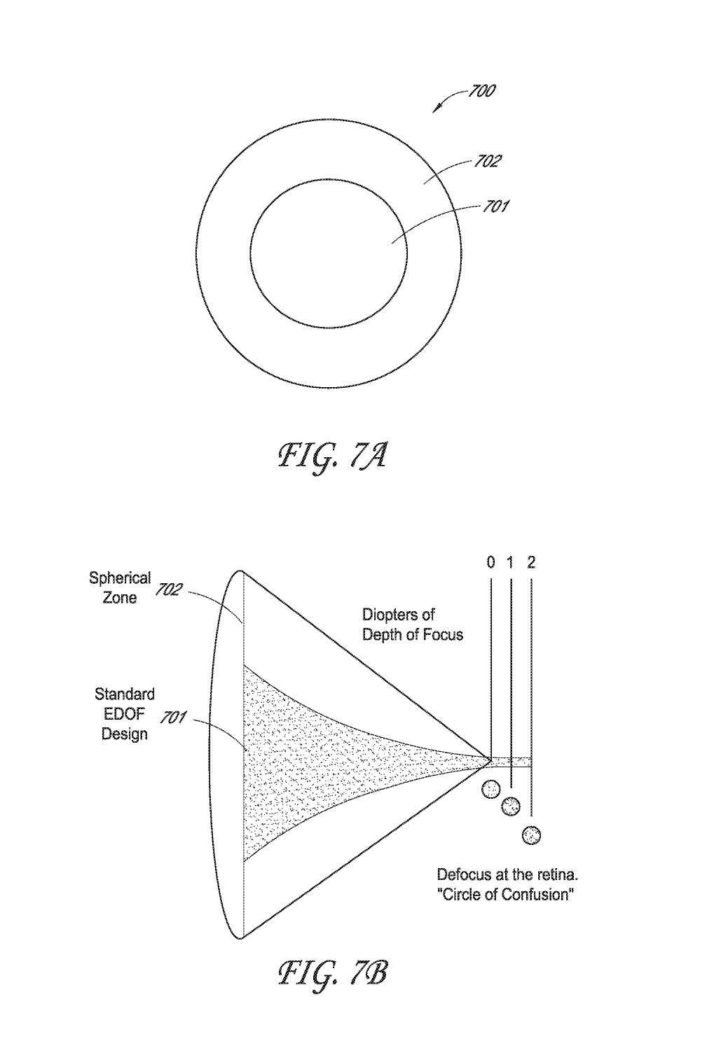

FIGS. 7A-7B are schematics for an example anterior surface and/or a posterior surface of such an optic. The anterior surface and the posterior surface can have a surface vertex. The optic can have an optical axis through the surface vertices. The anterior surface and/or a posterior surface of the example optic 700 can include a surface having a first portion 701 and a second portion 702. The first portion 701 can be configured to provide extended depth of field and the second portion 702 can be configured to provide monofocal distance correction and focusing. Referring to the defocus curves shown in FIG. 6C, the first portion 701 can have a defocus curve similar in shape to that of the “ideal” hyperfocal defocus curve, and the second portion 702 can have a defocus curve similar in shape to that of the standard spherical (monofocal) lens. Accordingly, the first portion 701 can be configured to provide extended depth of field, and the second portion 702 can be configured to provide enhanced distance vision or distance visual acuity. For example, the first portion 701 configured to provide an extended depth of field can supply near-equal visual acuity, or at least more than for the second portion 702, throughout a range of focus (e.g., far or distance, intermediate, near), while the second portion 702 can provide an enhanced vision quality metric for distance in comparison to the first portion 701. The enhanced vision quality metric can be a figure of merit for objects at distance (e.g., at or near 0.0 D). Objects between infinity and 2 meters (e.g., infinity to 2 meters, infinity to 3 meters, infinity to 4 meters, infinity to 5 meters, infinity to 6 meters, infinity to 7 meters, infinity to 8 meters, infinity to 9 meters, infinity to 10 meters, or any ranges in between any of these ranges) are considered distance. The figure of merit can be a modulation transfer function (MTF), a contrast sensitivity (CS), contrast, a derivation thereof, or a combination thereof. Other metrics can also be used to characterize image quality at the distance focus (which corresponds to the base power or labeled power of the lens) or for far objects. In some instances, the enhanced vision quality metric can be a higher value for the second portion 702 than for the first portion 701.

FIG. 7B illustrates how rays passing through the second portion 702 are focused on the distance vision focus (labeled as 0). (As referenced above, this distance vision. focus corresponds to the base power, labeled power, or distance power of the lens.) In contrast, rays passing through the first portion 701 form a caustic of near constant diameter through the far (0), intermediate (1), and near (2) foci as opposed to a single sharp focus at the distance (0) intermediate (1) or near (2) planes thereby providing an extended depth of field.

As shown in FIGS. 7A-7B, the first portion 701 can be disposed centrally within the optic 700. In some cases, the first portion is disposed centrally about the optical axis. The first portion 701 can have a maximum cross-sectional diameter in the range of about 2.5-4.5 mm (e.g., 2.5 mm, 2.75 mm, 3.0 mm, 3.25 mm, 3.5 mm, 3.75 mm, 4.0 mm, 4.25 mm, 4.5 mm, or any ranges between any of these sizes). Larger or smaller sizes may also be possible. The first portion 701 can have a surface profile as described herein with respect to optic 201 to provide extended depth of field. For example, the first portion 701 may introduce spherical aberration to provide extended depth of field. In some such examples, as described herein, the first portion 701 can have a shape comprising a conic or a biconic envelope offset by perturbations from the envelope comprising an aspheric higher order function of radial distance from the optical axis. Equation (2) describes an example shape using a conic term and even-powered polynomial terms. Other examples and combinations are possible. For example, the first portion 701 can have a shape comprising a biaspheric envelope. The biaspheric envelope can include two aspheric cross-sections in two orthogonal directions. In some instances, the biaspheric envelope can be offset by perturbations comprising an aspheric higher order function of radial distance from the optical axis.

The second portion 702 can surround the first portion 701. The second portion 702 can extend from the first portion 701 to the end of the optic 700. Accordingly, in some examples, the width of the second portion 702 can be the distance between the outer periphery of the first portion 701 to the edge of the optic 700. For example, the second portion 702 can have a width (e.g., a distance between inner and outer radii) in the range of about 1.0-3.5 mm (e.g., 1.0 mm, 1.25 mm, 1.5 mm, 1.75 mm, 2.0 mm, 2.25 mm, 2.5 mm, 2.75 mm, 3.0 mm, 3.25 mm, 3.5 mm, or any ranges between any of these sizes). Sizes outside these ranges are also possible.

The second portion 702 can have a different surface profile than the first portion 701. The first portion 701 can have higher spherical aberration control that provides extended depth of field than the second portion 702. In some cases, the second portion 702 may have substantially no spherical aberration control or at least no aberration control that provides extended depth of focus. For example, the second portion 702 can have a shape that comprises a conic, biconic, or biaspheric envelope not offset by perturbations comprising an aspheric higher order function of radial distance from the optical axis. In some cases, the second portion can have a shape that is spherical.

The second portion 702 can allow greater control of the marginal rays of the system such that a higher percentage of the rays that propagate through this portion are focused on the retina potentially providing increased contrast or improved vision quality as measure by other metrics for objects at a distance such as at infinity in comparison to the first portion (e.g., for distance power or labeled power of about +6 to −18 D). This allows a more defined focus for distance (possibly a smaller spot at the distance plane for distance objects), yet still provides the extended depth of field provided by the first portion 701. Thus, the second portion 702 can increase the responsivity distance vision quality, creating an improvement in focusing objects at a distance. This improved distance vision can be perceived by a patient as an increase in brain-favored “positive” metrics, e.g., contrast sensitivity (CS).

In addition, as the first portion 701 is configured to provide an extended depth of field, it can supply near-equal visual acuity or vision, or at least more than the second portion 702, throughout a range of focus (or for a range of object distances). The spot size, wavefront of the lens, and quality (e.g., as measured by a figure of merit such as MTF or CS) at distance, intermediate, and near points are substantially similar. However, this attribute can create difficulties in evaluating the power of the lens using standard metrology. Post-operative clinical evaluation of a patient using classical Gaussian metrology methods can also be challenging. Any number of focal points could be labeled and found to be a valid base power (e.g., distance or label power). In certain embodiments, the second portion 702 directing a ring of marginal rays to a distance focus location can provide a repeatable measurement more closely corresponding to distance power. Likewise, the second portion 702 can provide a benefit in determination of the classical base power of the implanted or un-implanted lens, and can assist in the ability to accurately measure the power of the lens using industry standard metrology methods. Thus, certain embodiments described herein can allow for standardized measurement of a lens with extended depth of field, including, but not limited to, negative-powered, positive-powered, toric, or any combination therein.

In various embodiments described herein, the first portion 701 can allow for the usage of different orders of spherical aberration and of a conic, biconic, or biaspheric base curve in order to balance the entire wavefront at each of its points near the exit pupil of the implanted eye, and the second portion 702 can allow for enhanced distance vision and/or monofocal distance focusing and for use of standard metrology.

In various embodiments, the anterior surface and/or posterior surface of the optic 700 can include other portions. For example, the anterior surface and/or the posterior surface of the optic 700 can further include a transition portion (not shown) providing a smooth transition without discontinuity between the first portion 701 and the second portion 702. The transition portion can also allow for additional wavefront optimization. In some embodiments, the transition portion can have a width (e.g., distance between the inner radii and the outer radii) in the range of about 0 to 1 mm (e.g., 0 mm, 0.1 mm, 0.2 mm, 0.3 mm, 0.4 mm, 0.5 mm, 0.6 mm, 0.7 mm, 0.8 mm, 0.9 mm, 1.0 mm, or any ranges between any of these sizes). Values outside these ranges are also possible. In some instances, the transition between the curvatures of the first portion 701 and the second portion 702 can be smooth enough that no transition region is desired.

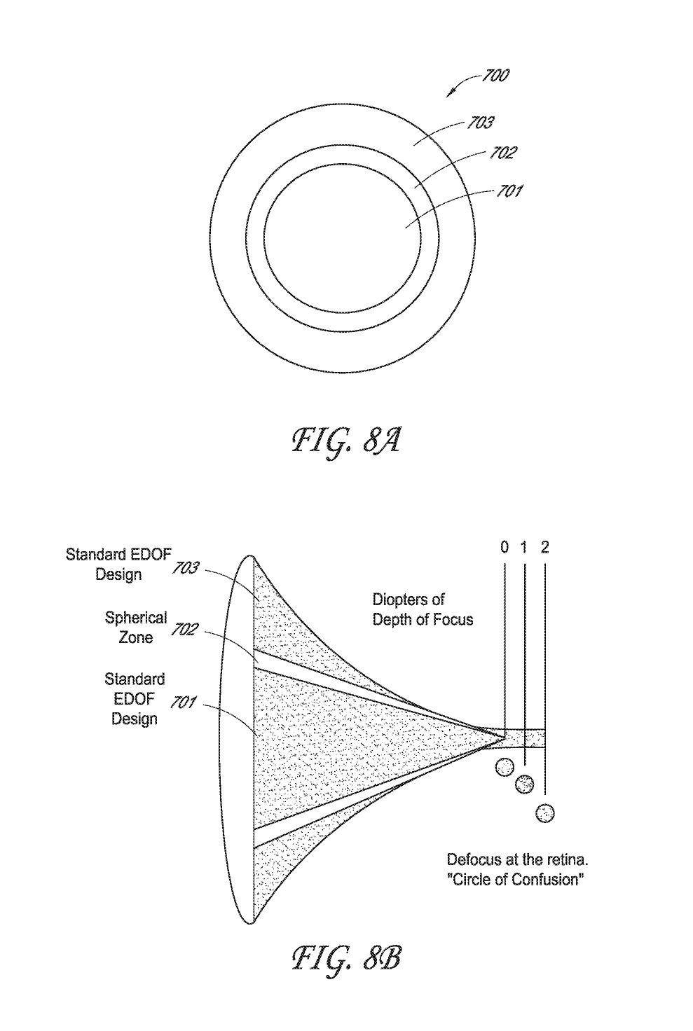

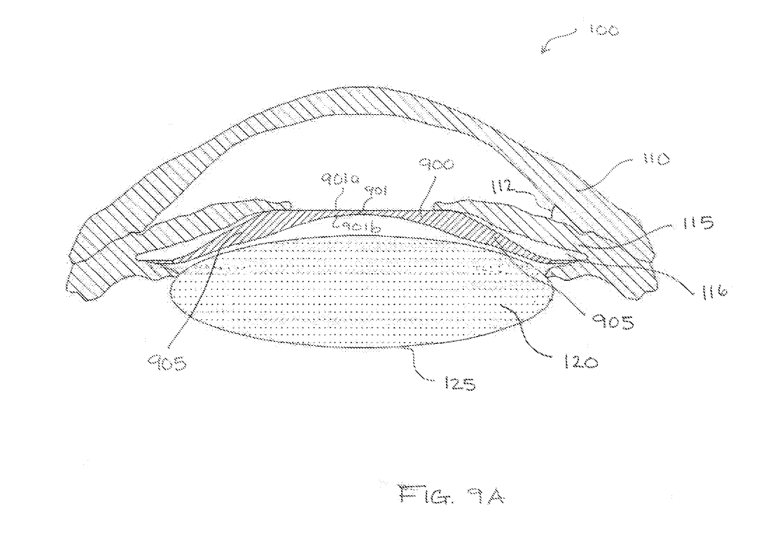

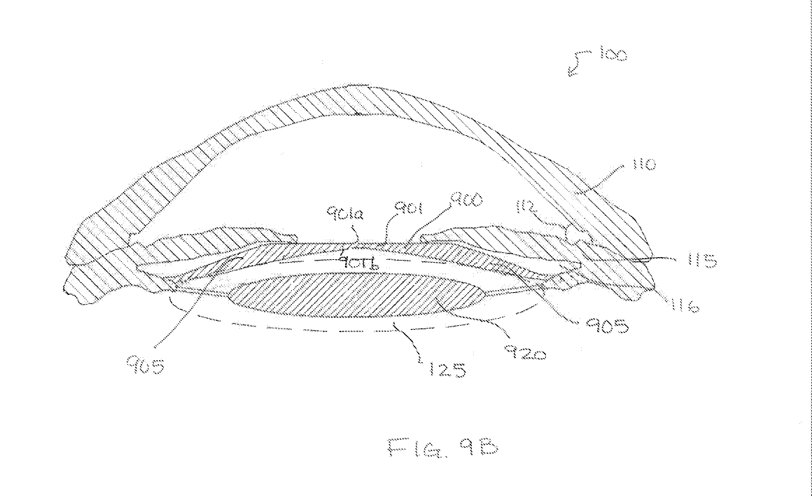

FIGS. 8A-8B are schematics for another example anterior surface and/or a posterior surface of an optic having a first portion configured to provide extended depth of field, and a second portion configured to provide enhanced distance visual acuity. In this example, the anterior surface and/or the posterior surface of the optic 700 can include a first onion 701 and a second portion 702 as in FIGS. 7A-7B. As shown in FIGS. 8A-8B. the anterior surface and/or the posterior surface of the optic 700 also can include a third portion 703 surrounding the second portion 702. In some such embodiments, the first portion 701 can have a maximum cross-sectional diameter in the range of about 2.5-4.5 mm (e.g., 2.5 mm, 2.75 mm, 3.0 mm, 3.25 mm, 3.5 mm, 3.75 mm, 4.0 mm, 4.25 mm, 4.5 mm, or any ranges between any of these sizes). The second portion 702 can be described as an annulus having a width between the inner and outer radii in the range of about 0.25-1.5 mm (e.g., 0.25 mm, 0.5 mm, 0.75 mm, 1.0 mm, 1.25 mm, 1.5 mm, or any ranges between any of these sizes). Furthermore, the third portion 703 can extend from the second portion 702 to the end of the optic 700. Accordingly, in some examples, the width of the third portion 703 can be the distance between the outer periphery of the second portion 702 to the edge of the optic 700. For example, the third portion 703 can have a width (e.g., distance between inner and outer radii) in the range of about 0.5-3.5 mm (e.g., 0.5 mm, 0.75 mm, 1.0 mm, 1.25 mm, 1.5 mm, 1.75 mm, 2.0 mm, 2.25 mm, 2.5 mm, 2.75 mm, 3.0 mm, 3.5 mm, or any ranges between any of these sizes). Values outside these ranges are also possible.

FIG. 8B illustrates how rays passing through the second portion 702 are focused on the distance vision focus (labeled as 0). In contrast, rays passing through the first portion 701 and third portion 703 focus continuously through the far (0), intermediate (1), and near (2) foci thereby providing an extended depth of field. As discussed above, the rays passing through the first portion 701 and third portion 703 form a caustic having nearly constant cross-section or beam diameter at the far (0), intermediate (1), and near (2) planes. This beam diameter, however, may potentially be larger than the size of the focus spot at the far image plane (0) formed by the rays propagating solely through of the second portion 702.

The third portion 703 can have a different surface profile than the second profile 702. For example, the third portion 703 can have higher spherical aberration control that provides extended depth of field than the second portion 702. In some examples, the third portion 703 can have a shape that comprises a conic, biconic, or biaspheric envelope offset by perturbations comprising an aspheric higher order function of radial distance from the optical axis.

In some embodiments, the third portion 703 can have a similar surface profile and/or substantially the same spherical aberration control as the first portion 701. For example, the third portion 703 can have substantially the same conic, biconic, or biaspheric envelope offset by perturbations with respect to the envelope comprising an aspheric higher order function of radial distance from the optical axis as the first portion.

As described herein, the first portion 701 and/or the third portion 703 can have a shape that comprises a conic, biconic, biaspheric envelope offset by perturbations comprising an aspheric higher order function of radial distance from the optical axis. In various embodiments, the aspheric higher order function can include at least one even order term, a₂ₙr²ⁿ, where n is an integer and a₂ₙ is a coefficient and r is the radial distance from the optical axis. For example, the aspheric higher order function can include a second order term, a₂r², where a₂ is a coefficient and r is the radial distance from the optical axis. The aspheric higher order function can include a fourth order term, a₄r⁴, where a₄ is a coefficient and r is the radial distance from the optical axis. The aspheric higher order function can also include a sixth order term, a₆r⁶ where a₆ is a coefficient and r is the radial distance from the optical axis. The aspheric higher order function can further include an eighth order term, a₈r⁸ where a₈ is a coefficient and r is the radial distance from the optical axis. The aspheric higher order function can include any combination of these higher order terms and possibly more terms.

In various embodiments, the anterior surface and/or the posterior surface of the optic 700 can further include a transition portion (not shown) providing a smooth transition without discontinuity between the second portion 702 and the third portion 703. The transition portion can also allow for additional wavefront optimization. In some embodiments, the transition portion can have a width (e.g., distance between the inner radii and the outer radii) in the range of about 0 to 1 mm (e.g., 0 mm, 0.1 mm, 0.2 mm, 0.3 mm, 0.4 mm, 0.5 mm, 0.6 mm, 0.7 mm, 0.8 mm, 0.9 mm, 1.0 mm, or any ranges between any of these sizes). Dimensions outside these ranges are also possible. In some instances, the transition between the curvatures of the second portion 702 and the third portion 703 can be smooth enough that no transition region is desired.

In some embodiments, the caustic of the second portion 702 can be sculpted to blend smoothly (or to provide a smoother transition) with the caustic of the first portion 701 and/or the caustic of the third portion 703. For example, as shown in FIG. 8B, the lower caustic envelope of the second portion 702 may not blend smoothly with the lower caustic envelope of the third portion 703 (e.g., see the discontinuity near the intersection of the caustics). Accordingly, in some embodiments, to provide a smoother caustic transition, the conic constant of the conic, biconic, or biaspheric envelope of the second portion 702 may be such to blend smoother with the caustic of the first portion 701 and/or the caustic of the third portion 703 (e.g., to fit more tightly with the ray envelope of the first portion 701 and/or to fit more tightly with the ray envelope of the third portion 703). For example, in some embodiments, the second portion 702 can have a conic constant such that the caustic of the second portion 702 blends smoothly with the caustic of the first portion 701, for example, more smoothly than if the second portion comprises a spherical surface. Furthermore, in some embodiments, the second portion 702 can have a conic constant such that the caustic of the second portion 702 blends smoothly with the caustic of the third portion 703, for example, more smoothly than if the second portion comprises a spherical surface. By having a smoother caustic transition, a slight misalignment in the surgical placement of the implants may be expected to produce a less noticeable effect on a patient's vision. In addition, with a smoother caustic transition, superimposed ghosting may potentially be reduced.

The various disclosures with respect to the optic 201 described herein can also apply to the various embodiments of FIGS. 7A-8B. For example, certain embodiments of FIGS. 7A-8B can be used for phakic or pseudophakic lens implants as described herein. In embodiments used for phakic lens implants, the optic 700 can have a thickness along the optical axis that is about 100-700 micrometers, about 100 to about 600 micrometers, about 100 to about 500 micrometers, about 100 to about 400 micrometers, about 100 to about 300 micrometers, or about 100 to about 200 micrometers (e.g., 100 micrometers, 200 micrometers, 300 micrometers, 400 micrometers, 500 micrometers, 600 micrometers, 700 micrometers, any value in between such ranges, or any range formed by such values). In embodiments for pseudophakic lens implants, the thickness along the optical axis can be about 700 micrometers to about 4 mm, about 700 micrometers to about 3 mm, about 700 micrometers to about 2 mm, about 700 micrometers to about 1 mm, any value in between such ranges, or any range formed by any values in these ranges. As another example, various embodiments of FIGS. 7A-8B can be used in a lens comprising at least one haptic disposed with respect to the optic 700 to affix the optic 700 in the eye when implanted therein. Furthermore, in some instances, the first portion 701 can be on the anterior surface of the optic, and the second portion 702 can be on the posterior surface of the optic. Likewise, in some instances, the first portion 701 can be on the posterior surface of the optic, and the second portion 702 can be on the anterior surface of the optic.

This disclosure also includes methods of implanting a lens into an eye of a patient, for example, a human being. These methods can be used to provide vision correction, e.g., of myopia, hyperopia, astigmatism, cataracts, and/or presbyopia. These methods can also be used to provide extended depth of field. The methods can implant, for example, any of the lenses described herein or lenses having any one or more features described herein. For example, the lens (e.g., lens 200 illustrated in FIG. 2) can include an optic (e.g., optics illustrated in FIGS. 4, 5A, 5B, 7A-7B, or 8A-8B) having an anterior and/or posterior surface configured to provide an extended depth of field and/or enhanced distance visual acuity.

As described herein, the lens can be inserted anterior of the natural lens of the eye such as a phakic intraocular lens. For example, FIG. 9A schematically illustrates a lens 900 inserted forward of the natural lens 120 in the eye 100. In some embodiments as illustrated in FIG. 9A, the lens 900 is inserted between the iris 115 and the natural lens 120. In some other embodiments (not shown), the lens can be inserted between the cornea 110 and the iris 115. As also described herein, the lens can be inserted in the capsular bag 125 in place of the natural lens 120 such as a pseudophakic intraocular lens. When a patient's vision is inferior to that expected after the implant procedure (e.g., not as good as desired or has worsened), replacement of the implanted lens may be desired. Replacement of a pseudophakic lens in the capsular bag 125 may have more risk of surgical complications than insertion and/or replacement of a phakic lens. Accordingly, FIG. 9B schematically illustrates an approach described herein that may utilize at least two artificial lenses, for example, a first artificial lens 900 implanted anterior of a second artificial lens 920 that is implanted in the capsular bag 125 such that the first anterior lens 900 can be replaced without having to replace the second lens 920 that is in the capsular bag 125.

With reference to FIG. 9A, various example lenses described herein may provide an amount of pressure on the sulcus 116 that is advantageously configured to provide a balance between stabilizing the lens 900 and reducing the likelihood of interfering with the proper regulation of the intraocular pressure in the eye 100. For example, as described herein, some embodiments can include one or more haptic portions 905. The haptic portions 905 can stabilize the lens 900 in the eye 100. For example, the haptic portions 905 can contact tissue in the eye 100 such as tissue in the sulcus 116 of the eye 100 posterior to iris 115 and provide frictional force that reduces movement of the lens 900 within the eye 100. In addition, the force exerted by tissue in the sulcus 116 can vault the lens (e.g., vault the haptic portions 905) toward the iris 115. Without being bound by theory, if little or no pressure were exerted (e.g., on the tissue in the eye 100, e.g., in the sulcus 116), a phakic intraocular lens may rotate and/or move and/or have relatively low vault. Such rotation and/or movement and/or low vault may cause the phakic lens to contact, rub against, scar, and/or damage the natural lens 120 (or possibly an intraocular lens replacing the natural lens), leading to light scattering sites which may lead to blurred vision and/or glare (e.g., cortical cataracts). Such rotation and/or movement may also cause the phakic lens to contact or rub against the iris and this can cause pigment cells to slough off from the back of the iris and float around in the aqueous humor. These cells can accumulate in the trabecular meshwork and block drainage from the aqueous humor thus resulting in increased pressure in the eye that may lead to eye injury and damage, such as glaucoma (e.g., damage to the optic nerve). In some cases, iris pigment dispersion may cause the patient to have an iridectomy. Rotation of a toric phakic lens (or possibly an intraocular lens replacing the natural lens) may also provide less than desirable results if the prescribed angle of the cylinder axis is not retained.

Too much exerted pressure, however, may also lead to eye injury and damage. For example, the intraocular pressure can be regulated by the flow of intraocular fluid (e.g. aqueous humor). The fluid can be produced in the eye and drained, for example, through the angle 112 between the iris 115 and the cornea 110. An intraocular lens exerting too much pressure on the sulcus 116 can result in too much vaulting and pressure on the iris 115. Such pressures on the iris 115 may narrow the angle 112, which may reduce and/or block the flow of the intraocular fluid causing an increase in intraocular pressure in the eye 100. High intraocular pressure may lead to eye injury and damage, such as glaucoma (e.g., damage to the optic nerve). Various designs described herein can provide sufficient pressure on the tissue of the eye 100, e.g., sulcus 116, that advantageously stabilizes the lens 900 in the eye 100 and reduces instances of increasing intraocular pressure caused by too much pressure exerted on the sulcus 116 and/or other tissue of the eye 100.

With reference to FIG. 9B, this application also discloses two artificial lenses 900, 920 being implanted in the eye 100 of a patient. In some cases, the first artificial lens 900 (supplementary intraocular lens) can be implanted anterior of a second artificial lens 920 that is implanted in the capsular bag 125. For example, the first anterior lens 900 can be positioned between the iris 115 and the second lens 920.

Certain embodiments of the supplementary intraocular lens (e.g., first artificial lens 900) illustrated in FIG. 9B can provide advantages over some embodiments of the phakic lens illustrated in FIG. 9A. For example, in some designs of the phakic lens shown in FIG. 9A, balance between too low vault and too high intraocular pressure can be provided. In some embodiments of the supplementary intraocular lens shown in FIG. 9B, there can be reduced concern over too low vault. For example, in some examples, the second artificial lens 920 can be provided to sit lower than the natural lens 120 such that the supplementary intraocular lens 900 can also be provided to sit lower than a phakic lens. In addition, in some embodiments, contact between two artificial lenses can be less troublesome as contact (e.g., rubbing against one another) can be less likely to cause cataracts or other serious optical effects. Accordingly, various embodiments of a supplementary intraocular lens can be configured to provide sufficient force to stabilize the lens yet may be allowed to sit lower than a phakic lens (e.g., can have lower vault and/or sit lower in depth within the eye/closer to the retina). Therefore, in some embodiments, the sagitta of the supplementary lens (prior to insertion) can be smaller compared to the sagitta of a phakic lens.

As illustrated in FIG. 9A, the phakic lens 900 is inserted forward of the natural lens 120. The phakic lens 900 vaults above the horizontal plane of the sulcus 116 towards the iris 115 (e.g., to reduce contact with the natural lens 120). As illustrated, the phakic lens 900 can exert pressure on the iris 115 causing the angle 112 between the cornea 110 and the iris 115 to narrow and possibly leading to increased intraocular pressure. In some instances, in order to make space for the phakic lens 900 between the iris 115 and the natural lens 120, carbon dioxide may be pumped into the eye to move the iris 115 forward, which may also narrow the angle 112 and increase intraocular pressure.

As illustrated in FIG. 9B, the supplementary intraocular lens 900 is inserted forward a second artificial lens 920. In some embodiments, the second artificial lens 920 can sit lower than the natural lens 120 and the supplementary intraocular lens 900 can also sit lower, reducing the pressure on the iris 115. As such, in some embodiments, the supplementary intraocular lens 900 can be relatively flat, e.g., prior to insertion into the eye. Accordingly, various embodiments of the supplementary intraocular lens 900 can fit into the sulcus 116 with less vault above the horizontal plane of the sulcus 116 (e.g., having a distance of 0.0 mm to 0.5 mm, 0.0 mm to 0.75 mm, 0.0 mm to 1 mm, etc. from the center of the optic to the plane of the sulcus 116). In some instances, the posterior surface of the supplementary intraocular lens 900 can be substantially level with the plane of the sulcus 116. In addition, in many instances, the supplementary intraocular lens 900 can be inserted without having to pump carbon dioxide into the eye. Thus, in many embodiments, the iris 115 can rest in a natural or approximately natural position without interfering with the angle 112 between the iris 115 and the cornea 110.

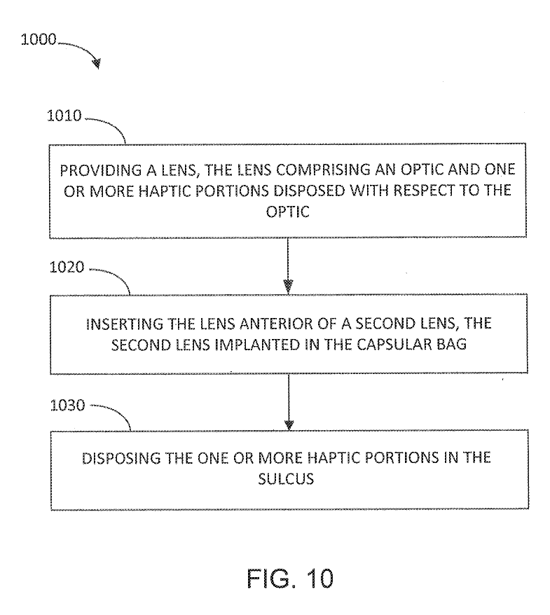

FIG. 10 schematically illustrates an example method 1000 of implanting the first anterior lens 900 into an eye 100 of a patient. The method 1000 can include providing a lens 900 as shown in operation block 1010. The method 1000 can include placing the lens 900 anterior of a second artificial lens 920 that is implanted in the capsular bag 125 as shown in operation block 1020. As shown in operation block 1030, the method 1000 can also include disposing one or more haptic portions 905 in the sulcus 112 and possibly contacting other tissue in the eye.

With reference to operation block 1010, the method 1000 can include providing a lens 900. The lens 900 can include any of the lenses as described herein. For example, with reference to FIG. 9B, the lens 900 can include an optic 901 and one or more haptic portions 905 disposed about the optic 901. The optic 901 can include transparent material such as a collagen copolymer material (e.g., similar to material used in Collamer® IOLs by STAAR® Surgical Company), a hydrogel, a silicone, and/or an acrylic. The optic 901 can have an anterior surface 901a and a posterior surface 901b. As described herein, the anterior surface 901a and/or the posterior surface 901b can include an aspheric surface, e.g., to provide an extended depth of field and/or enhanced distance vision acuity.

This application describes how the lens can include anterior and/or posterior surfaces shaped to provide a radial power profile characterized by Φ(r)=a+br²+cr⁴+dr⁶+er⁸ for wavefront at the exit pupil of the optic for an object vergence of 0 to 2.5 Diopter (D) where r is the radial distance from the optical axis and a, b, c, d, and e are coefficients. In some designs, the anterior surface can have an aspheric shape that comprises a conic or biconic offset by perturbations comprising an aspheric higher order function of radial distance from the optical axis. For example, as described herein, the aspheric higher order function can include at least one even order term, a₂ₙr²ⁿ, where n is an integer and a₂ₙ is a coefficient and r is the radial distance from the optical axis. For example, the aspheric shape can be described using the conic term and the even-powered polynomial terms (e.g., describing an even asphere) as shown in example equation (2) described above. As can be seen in the example equation (2), the higher order function can include at least a second order term (a₂r²), a fourth order term (a₄r⁴), a sixth order term, (a₆r⁶), and/or an eighth order term (a₈r⁸). In some embodiments, the higher order function can include one or more odd order terms. For example, the higher order function can include only odd order terms or a combination of even and odd order terms. In some examples, the anterior surface can have an aspheric shape that comprises a biconic offset by the perturbations. In some embodiments, the posterior surface can have an aspheric shape that comprises a biconic offset by perturbations comprising an aspheric higher order function of radial distance from the optical axis. In some such embodiments, the posterior surface can have an absolute value of ratio Rₓ/R_{y} between 0 and 100 (e.g., between 0 and 75, between 0 and 50, between 0 and 25, between 0 and 10, between 0.1 and 10, between 0.2 and 10, between 0.25 and 10, between 0.5 and 10, etc.) and an absolute value of ratio kₓ/k_{y} between 0 and 100 (e.g., between 0 and 75, between 0 and 50, between 0 and 25, between 0 and 10, between 0.1 and 10, between 0.2 and 10, between 0.25 and 10, between 0.5 and 10, etc.). As described herein, Rₓ can be the radius of the surface in the x direction (e.g., the inverse of the curvature in the x direction), R_{y} can be the radius of the surface in the y direction (e.g., the inverse of the curvature in the y direction), kₓ can be the conic constant for the x direction, and k_{y} can be the conic constant for the y direction. Other examples as described herein are also possible. Moreover, this lens 900 can include any lens feature described herein such as features relating to shape, material, optical design parameter, etc., as well as any combination of any lens features disclosed herein.

With reference to operation block 1020 in FIG. 10, the method 1000 can include placing the lens 900 anterior of a second lens 920 implanted in the capsular bag 125. For example, as described herein and shown in FIG. 9B, the lens 900 can be inserted between the iris 115 and the second lens 920. As another example, the lens 900 can be inserted between the cornea 110 and the iris 115.

In some embodiments, the posterior artificial lens 920 can be implanted in the capsular bag 125. For example, the method can include forming an opening in tissue of the eye 100 and inserting the posterior artificial lens 920 in the capsular bag 125. In some embodiments, the lens 920 in the capsular bag 125 may have already been inserted in the capsular bag 125 such as from a previous implant procedure. As an example, a patient's vision may be less than satisfactory or may have changed (e.g., worsened) after a previous procedure. Instead of removing the previously implanted lens 920 in the capsular bag 125, artificial lens 900 may be implanted anterior of the previously implanted lens 920 to help correct the unsatisfactory or changed vision. The posterior artificial lens 920 can include any of the lenses disclosed herein or any other pseudophakic lens known in the art or yet to be developed. Moreover, the posterior artificial lens 920 can include any lens feature described herein such as features relating to shape, material, optical design parameter, etc., as well as any combination of any lens features disclosure herein.

In various situations, the anterior lens 900 can be used in conjunction with the second artificial lens 920 to provide vision correction. For example, the combination of first anterior artificial lens 900 and the second artificial lens 920 together may provide optical power for imaging an object onto the retina. In some examples, first artificial lens 900 may be configured to provide a first aspect of vision correction, and the second artificial lens 920 may be configured to provide a second aspect of vision correction. For example, the second lens 920 may be configured to provide optical power to correct myopia, hyperopia, while the anterior artificial lens 900 may be configured to provide extended depth of field (or vice versa). The anterior artificial lens 900 and/or the second artificial lens 920 may provide astigmatism correction and thus may have cylinder. Accordingly, the anterior artificial lens 900 and/or the second artificial lens 920 may comprise a toric lens such as for example the toric lenses described herein or other lens shape the includes cylinder. Cylinder in the amount between +0.5 D and +10 D are possible. The cylinder axes can be in any orientation, e.g., 0° to 360°. In some instances, second artificial lens 920 may improve distance vision, such as monofocal distance focusing. The anterior lens 900 may have 0 dioptric power and be configured to provide enhanced depth of field. Other combinations of vision correction are possible. For example, the anterior lens 900 may provide 0.25 to 0.75 or 0.2 to 1.0 Diopter power. The anterior lens 900 may provide high power as well. For example, the anterior lens 900 may provide 1.0 to 5.0, 1.0 to 10.0, 1.0 to 15.0, or 1.0 to 20.0 Diopter power. The anterior lens 900 may have other powers such as other power values described herein as well as power values not specifically recited herein.

In some examples, the second artificial lens 920 may have already been implanted in the capsular bag 125. The second lens 920 may be configured to provide monofocal focusing. In some such embodiments, the anterior lens 900 may be configured to provide monofocal focusing, e.g., to correct residual refraction. In some other embodiments, the anterior lens 900 may be configured to provide extended depth of field or multifocal focusing, and potentially to correct residual refraction.

In some examples, the already implanted second artificial lens 920 may be configured to provide extended depth of field and the anterior lens 900 may be configured to provide monofocal focusing, e.g., to correct residual refraction. Alternatively, the already implanted second artificial lens 920 and the anterior lens 900 may both be configured to provide extended depth of field.

In some examples, the second artificial lens 920 may be implanted in the capsular bag 125 to provide monofocal focusing and immediately or shortly afterwards (e.g., the same surgical procedure, the same visit, the same day), the anterior lens 900 may be implanted to provide extended depth of field, e.g., to provide improved range of vision. In some such instances (e.g., for cataract surgery), diagnostic instrumentations such as intraoperative wavefront sensing, e.g., Optiwave Refractive Analysis (ORA) system, can be used to determine what additional correction is to be provided, e.g., by the anterior lens 900. In cases of poor power targeting or patient dissatisfaction with vision, for example, the anterior lens 900 can be exchanged or removed more easily than replacement of the artificial lens 920 in the capsular bag 125. In some examples, the second artificial lens 920 may be implanted in the capsular bag 125 to provide extended depth of field and immediately or shortly afterwards (e.g., the same surgical procedure, the same visit, the same day), the anterior lens 900 may be implanted to provide monofocal focusing.

One aspect of the disclosure herein is a method of treating cataracts or presbyopia by providing extended depth of field focusing to provide extended depth of field vision in a patient, where the method comprises, in a patient in which a first artificial lens has been positioned in an eye to replace a native crystalline lens, and during a patient visit in which the first artificial lens was positioned in the eye, implanting a second artificial lens into the eye in a position that is anterior to the first artificial lens, the second artificial lens configured to provide extended depth of field focusing, wherein the second artificial lens includes an optic portion and one or more haptic portions extending peripherally from the optic portion, the optic portion being transparent and having an anterior surface and a posterior surface, and at least one of the anterior and posterior surfaces comprises an aspheric surface. Lens 920 is an example of a first artificial lens according to this method, and lens 900 is an example of a second artificial lens according to this method. The method can be performed to treat a patient with cataracts, but the method can also be used treat a patient that has presbyopia but does not have cataracts.

In some examples, the first artificial lens 900 and/or the second artificial lens 920 may be configured to provide both monofocal focusing and extended depth of field. For example, the first artificial lens 900 and/or the second artificial lens 920 may have lens portions that provide for different aspects of vision correction (e.g., FIGS. 7A-7B or 8A-8B). Other examples are possible.

With reference to operation block 1030 in FIG. 10, the method 1000 can also include contacting the one or more of the haptic portions 905 to tissue within the eye such as tissue in the sulcus. In some examples, with reference to FIG. 9B, one or more haptic portions 905 can be contacted to and/or affixed to the sulcus 116 and/or other tissue in the eye 100 such that contact between the anterior lens 900 and second artificial lens 920 in the capsular bag can be reduced. Rotation and/or movement and/or low vault of the anterior intraocular lens may cause contact with the posterior lens. In some instances rotation of the lens may cause problems such as not retaining the angle of the cylinder axis of a toric lens. In some instances, contact between two intraocular lenses may potentially lead to damaged areas of one or both of the lenses, which may cause blurred vision and/or glare due to the light scattering sites and higher order aberrations induced to optical surfaces of the lenses. Such rotation and/or movement may also cause the anterior lens to contact or rub against the iris such that pigment cells from the iris might block drainage from the aqueous humor and increase the intraocular pressure in the eye.

In certain embodiments, exerting pressure on the sulcus 116 or other tissue in the eye can help stabilize lens 900 in the eye 100. The length of the anterior lens 900, measured from the ends of the haptics portions 905 may be slightly larger than the area in which the anterior lens 900 is inserted in the eye 100. The haptic portions 905 may likewise contact tissue in the eye 100 such as tissue in the sulcus 116. The force of this contact may create a force normal and a frictional force (generally characterized by the force normal multiplied by a coefficient of friction) that can reduce movement of the anterior lens 900 within the eye 100. Accordingly, by exerting pressure on the sulcus 116 or other tissue in the eye 100, some designs can reduce harmful contact between the anterior lens 900 and second lens 920. For example, exerting pressure on the sulcus 116 or other tissue in the eye 100 can reduce rotation and/or movement of some designs of the anterior lens 900.

While exerting some pressure on tissue in the eye can advantageously help stabilize the anterior lens 900 in the eye and reduce chances of contact between two lenses, exerting too much pressure may be problematic in some instances. For example, a lens 900 exerting high pressure on the iris 115 (e.g., from high vault) may narrow the angle 112 between the iris 115 and the cornea 110. Narrowing the angle 112 may inhibit the flow of intraocular fluid thereby increasing the intraocular pressure, leading to eye injury and/or damage, such as damage to the optic nerve.

Various designs described herein can provide a pressure on the sulcus 116 and/or other tissue in the eye 100 that is advantageously configured to provide a balance between stabilizing the anterior lens 900 and permitting normal regulation of the intraocular pressure in the eye 100. For example, the exerted pressure on the sulcus 116 can be in a range from about 0.1 N to about 1.0 N, or any range within this range (e.g., from about 0.2 N to about 1.0 N, from about 0.3 N to about 1.0 N, from about 0.4 N to about 1.0 N, from about 0.5 N to about 1.0 N, from about 0.1 N to about 0.9 N, from about 0.1 N to about 0.8 N, from about 0.1 N to about 0.7 N, from about 0.1 N to about 0.6 N, from about 0.1 N to about 0.5 N, etc.), or any ranges formed by any combination of these ranges or values. In some cases, such pressures exerted on the sulcus 116 or other ocular tissue can help stabilize the anterior lens 900. Additionally, in some cases, some such pressures exerted on the sulcus 116 or other ocular tissue can help reduce the amount of tissue movement, which can help prevent narrowing of the angle 112 between the iris 115 and the cornea 110.

In some examples, as described herein, the haptic portions 905 may be sized to provide suitable vaulting (without exerting too much pressure on the iris 115) to increase clearance between the optics of anterior lens 900 and second lens 920 in the capsular bag. The length of the lens from the end of one haptic portion 905 to the end of the other haptic portion 905 may be larger than the space in the eye into which it is inserted. However, this length may not be so much larger to induce an amount of vaulting of the lens 900 and optic 901 that causes the anterior lens 900 to contact the second lens 920 and/or exert high pressure on the iris 115. For some designs, the optic of one of the two lenses 900, 920 may comprise a collagen copolymer, a hydrogel, a silicone, and/or an acrylic. Some such materials may advantageously allow contact with another lens and/or parts of the eye 100 with reduced and/or substantially no damage.

In some cases, because the anterior lens 900 is positioned forward of another artificial lens 920 (e.g., not the natural crystalline lens 120), some contact between the lenses may be permissible. Accordingly, for some designs, the anterior lens 900 can be thicker at the center of the optic 901 compared to lenses that may be placed forward of the natural crystalline lens 120. For example, the anterior surface 901a and the posterior surface 901b of the anterior lens 900 can have a surface vertex. The optic 901 can have an optical axis through the surface vertices and a thickness along the optical axis. For some designs, the thickness along the optical axis can be in the range from about 100 micrometers to about 2 mm, or any range within this range (e.g., from about 100 micrometers to about 1 mm, from about 100 micrometers to about 1.5 mm, from about 200 micrometers to about 1.7 mm, from about 200 micrometers to about 2 mm, from about 300 micrometers to about 1.7 mm, from about 300 micrometers to about 2 mm, from about 400 micrometers to about 1.7 mm, from about 400 micrometers to about 2 mm, from about 500 micrometers to about 1.7 mm, from about 500 micrometers to about 2 mm, etc.), or any ranges formed by any combination of these ranges or values.

or reasons described herein, in various cases, an anterior lens 900, prior to insertion into the eye with an artificial lens 920 in the capsular bag 125, can be relatively flat compared to an anterior lens 900 to be inserted in the eye having the natural lens 120 in place. For example, the distance as measured along the optical axis between the posterior surface of the lens and a plane defined by the ends of the haptic farthest from the optical axis can between 0.5 mm and 0.0 mm. This distance may be larger in other designs as well.

In some examples, the anterior surface 901a of the optic 901 of anterior lens 900 can be convex, which may reduce and/or prevent chaffing of or tissue damage to the iris 115. In some other instances, the anterior surface 901a of the optic 901 of lens 900 can be substantially flat. In some instances, the anterior surface 901a of the optic 901 of lens 900 can be concave.

In some examples, the posterior surface 901b of the optic 901 of anterior lens 900 can be concave to help reduce and/or prevent damage with the second lens 920. For example, the anterior surface 901a of the optic 901 can be convex, and the posterior surface 901b of the optic 901 can be concave such that the optic 901 is meniscus shaped. As described herein, being positioned forward of another artificial lens 920, some designs of the lens 900 may allow for some contact between the lenses 900, 920. In certain instances, the posterior surface 901b of the optic 901 of lens 900 can be substantially flat or convex. For example, the anterior surface 901a of the optic 901 of anterior lens 900 can be convex, and the posterior surface 901b of the optic 901 can be substantially flat such that the optic 901 is plano-convex.

As another example, the anterior surface 901a of the optic 901 of anterior lens 900 can be convex, and the posterior surface 901b of the optic 901 can be convex such that the optic 901 is biconvex. Other examples and shapes are possible.

If vision changes or is not satisfactory after implantation of the anterior lens 900, in certain cases replacement of lens 900 is possible without replacement of the second lens 920 in the capsular bag 125. In some embodiments, a new lens 900 can be inserted into the eye 100 without removal of the lens 920 in the capsular bag 125. For example, the new anterior lens 900 can be inserted anterior of the already implanted lens 920. The combination of the new anterior lens 900 and the previously implanted lens 920 together may provide updated vision correction.

The terms “about” and “substantially” as used herein represent an amount equal to or close to the stated amount (e.g., an amount that still performs a desired function or achieves a desired result). For example, unless otherwise stated, the terms “about” and “substantially” may refer to an amount that is within (e.g., above or below) 10% of, within (e.g., above or below) 5% of, within (e.g., above or below) 1% of, within (e.g., above or below) 0.1% of, or within (e.g., above or below) 0.01% of the stated amount.

Various embodiments of the present invention have been described herein. Although this invention has been described with reference to these specific embodiments, the descriptions are intended to be illustrative of the invention and are not intended to be limiting. Various modifications and applications may occur to those skilled in the art without departing from the true spirit and scope of the invention.

### Additional Examples

1. A method of implanting a first lens into an eye of a human, the method comprising:
providing the first lens; and
inserting the first lens anterior of a second lens, wherein the second lens comprises an artificial lens, and wherein at least one of the first and second lenses comprises an optic and one or more haptic portions disposed about the optic, the optic comprising transparent material, the optic having an anterior surface and a posterior surface, and at least one of the anterior and posterior surfaces comprising an aspheric surface.

2. The example of example 1, wherein the at least one of the first and second lenses comprises the first lens.

3. The example of example 2, wherein the first lens is configured to provide multifocal focusing and the second lens is configured to provide monofocal focusing.

4. The example of any of the preceding examples, wherein the first lens is configured to provide an extended depth of field.

5. The example of example 1, wherein the at least one of the first and second lenses comprises the second lens.

6. The example of example 5, wherein the first lens is configured to provide monofocal focusing and the second lens is configured to provide multifocal focusing.

7. The example of any of the preceding examples, wherein the second lens is configured to provide an extended depth of field.

8. The example of example 1, wherein the at least one of the first and second lenses comprises the first and second lenses.

9. The example of example 8, wherein the first and second lenses are configured to provide multifocal focusing.

10. The example of any of the preceding examples, wherein the first and second lenses are configured to provide an extended depth of field.

11. The example of example 1, wherein the first and second lenses are configured to provide monofocal focusing.

12. The example of any of the preceding examples, wherein the anterior surface is convex.

13. The example of any of the preceding examples, wherein the posterior surface is concave.

14. The example of example 12, wherein the posterior surface is concave such that the optic is meniscus shaped.

15. The example of any of examples 1-10 or 12-14, wherein the at least one of the first and second lenses has 0 dioptric power.

16. The example of any of the preceding examples, wherein the transparent material comprises collamer.

17. The example of any of examples 1-15, wherein the transparent material comprises silicone, acrylic, or hydrogel.

18. The example of any of the preceding examples, wherein the anterior and posterior surfaces are shaped to provide a radial power profile characterized by Φ(r)=a+br²+cr⁴+dr⁶+er⁸ for wavefront at an exit pupil of the optic for an object vergence of 0 to 2.5 Diopter (D), where r is the radial distance from the optical axis and a, b, c, d, and e are coefficients.

19. The example of any of the above examples, wherein the anterior surface has an aspheric shape that comprises a conic or biconic offset by perturbations comprising an aspheric higher order function of radial distance from the optical axis.

20. The example of example 19, wherein said aspheric higher order function includes a second order term, a₂r², where a₂ is a coefficient and r is the radial distance from the optical axis.

21. The example of examples 19 or 20, wherein said aspheric higher order function includes a fourth order term, a₄r⁴, where a₄ is a coefficient and r is the radial distance from the optical axis.

22. The example of any of examples 19-21, wherein said aspheric higher order function includes a sixth order term, a₆r⁶ where a₆ is a coefficient and r is the radial distance from the optical axis.

23. The example of any of examples 19-22, wherein said aspheric higher order function includes an eighth order term, a₈r⁸ where a₈ is a coefficient and r is the radial distance from the optical axis.

24. The example of example 19, wherein the aspheric higher order function includes at least one even order term, a₂ₙr²ⁿ, where n is an integer and a₂ₙ is a coefficient and r is the radial distance from the optical axis.

25. The example of any of examples 19-24, wherein the anterior surface has an aspheric shape that comprises a biconic offset by said perturbations.

26. The example of any of the preceding examples, wherein the posterior surface has an aspheric shape that comprises a biconic offset by perturbations comprising an aspheric higher order function of radial distance from the optical axis, and wherein the posterior surface has an absolute value of ratio Rₓ/R_{y} between 0 and 100 and an absolute value of ratio kₓ/k_{y} between 0 and 100.

27. The example of any of the preceding examples, wherein the anterior and posterior surfaces comprise aspheric surfaces.

28. The example of any of the preceding examples, wherein the one or more haptic portions comprise a plurality of haptic portions.

29. The example of any of the preceding examples, wherein the anterior surface and the posterior surface have a surface vertex, the optic having an optical axis through the surface vertices and a thickness along the optical axis that is in a range from about 100 micrometers to about 2 mm.

30. The example of any of the preceding examples, wherein the one or more haptic portions contact the sulcus with a pressure in a range from about 0.1N to about 1.0N.

31. The example of any of the preceding examples, wherein inserting the first lens comprises inserting the first lens between the iris and the second lens.

32. The example of any of examples 1-30, wherein inserting the first lens comprises inserting the first lens between the cornea and the iris.

33. The example of any of the preceding examples, wherein the second lens is in the capsular bag.

34. The example of any of examples 1-12 or 15-33, wherein the posterior surface is substantially flat.

35. The example of example 12, wherein the posterior surface is substantially flat such that the optic is plano-convex.

36. The example of any of the preceding examples, wherein the posterior surface of the first lens is substantially level with the plane of the sulcus.

37. The example of any of the preceding examples, wherein the iris rests in an approximately natural position.

What is claimed is:

1. A method of treating cataracts or presbyopia by providing extended depth of field focusing to provide extended depth of field vision in a patient, comprising:
in a patient in which a first artificial lens has been positioned in an eye to replace a native crystalline lens, and during a patient visit in which the first artificial lens was positioned in the eye,
implanting a second artificial lens into the eye in a position that is anterior to the first artificial lens, the second artificial lens configured to provide extended depth of field focusing,
wherein the second artificial lens includes an optic portion and one or more haptic portions extending peripherally from the optic portion, the optic portion being transparent and having an anterior surface and a posterior surface, and at least one of the anterior and posterior surfaces comprises an aspheric surface.

2. The method of claim 1, wherein the first artificial lens that has been positioned in the eye is configured to provide monofocal focusing.

3. The method of claim 1, wherein the first artificial lens has been positioned in a capsular bag.

4. The method of claim 1, wherein implanting the second artificial lens comprising implanting the second artificial lens posterior to an iris of the eye.

5. The method of claim 1, wherein the posterior surface of the second artificial lens has an aspheric shape that comprises a biconic offset by perturbations comprising an aspheric higher order function of radial distance from the optical axis, and wherein the posterior surface has an absolute value of ratio Rx/Ry between 0 and 100 and an absolute value of ratio kx/ky between 0 and 100.

6. The method of claim 1, wherein the anterior surface of the second artificial lens has an aspheric shape that comprises a biconic offset by perturbations comprising an aspheric higher order function of radial distance from the optical axis, and wherein the anterior surface has an absolute value of ratio Rx/Ry between 0 and 100 and an absolute value of ratio kx/ky between 0 and 100.

7. The method of claim 1, wherein the anterior surface of the second artificial lens is convex.

8. The method of claim 1, wherein the posterior surface of the second artificial lens is concave.

9. The method of claim 8, wherein the posterior surface is concave such that the optic is meniscus shaped.

10. The method of claim 1, wherein at least one of the first and second lenses has 0 dioptric power.

11. The method of claim 1, wherein the transparent material comprises collamer.

12. The method of claim 1, wherein the transparent material comprises at least one of silicone, acrylic, and hydrogel.

13. The method of claim 1, wherein the anterior and posterior surfaces of the second artificial lens are shaped to provide a radial power profile characterized by Φ(r)=a+br2+cr4+dr6+er8 for wavefront at an exit pupil of the optic for an object vergence of 0 to 2.5 Diopter (D), where r is the radial distance from the optical axis and a, b, c, d, and e are coefficients.

14. The method of claim 1, wherein the anterior surface has an aspheric shape that comprises a conic or biconic offset by perturbations comprising an aspheric higher order function of radial distance from the optical axis.

15. The method of claim 14, wherein the aspheric higher order function includes a second order term, a2r2, where a2 is a coefficient and r is the radial distance from the optical axis.

16. The method of claim 15, wherein the aspheric higher order function includes a fourth order term, a4r4, where a4 is a coefficient and r is the radial distance from the optical axis.

17. The method of claim 16, wherein the aspheric higher order function includes a sixth order term, a6r6 where a6 is a coefficient and r is the radial distance from the optical axis.

18. The method of claim 17, wherein the aspheric higher order function includes an eighth order term, a8r8 where a8 is a coefficient and r is the radial distance from the optical axis.

19. The method of claim 14, wherein the aspheric higher order function includes at least one even order term, a2nr2n, where n is an integer and a2n is a coefficient and r is the radial distance from the optical axis.

20. The method of claim 14, wherein the anterior surface has an aspheric shape that comprises a biconic offset by said perturbations.

21. The method of claim 1, wherein the anterior and posterior surfaces of the second artificial lens comprise aspheric surfaces.

22. The method of claim 1, wherein the anterior surface and the posterior surface each have a surface vertex, the optic having an optical axis through the surface vertices and a thickness along the optical axis that is in a range from about 100 micrometers to about 2 mm.

23. The method of claim 1, wherein implanting the second artificial lens into the eye comprises the one or more haptic portions contacting a sulcus of the eye with a pressure in a range from about 0.1 N to about 1.0 N.

24. The method of claim 1, wherein the anterior surface of the second artificial lens is substantially flat.

25. The method of claim 24, wherein the anterior surface of the second artificial lens is substantially flat such that the optic is plano-convex.

26. The method of claim 1, wherein implanting the second artificial lens comprising implanting the second artificial lens such that the posterior surface of the second artificial lens is substantially level with the plane of a sulcus of the eye.

27. The method of claim 1, wherein after implanting the second artificial lens, an iris of the eye rests in an approximately natural position.

28. A method of treating cataracts or presbyopia by providing multifocal focusing to provide multifocal vision in a patient, comprising:
in a patient in which a first artificial lens has been positioned in an eye to replace a native crystalline lens, and during a patient visit in which the first artificial lens was positioned in the eye, implanting a second artificial lens into the eye in a position that is anterior to the first artificial lens, the second artificial lens configured to provide multifocal focusing,
wherein the second artificial lens includes an optic portion and one or more haptic portions extending peripherally from the optic portion, the optic portion being transparent and having an anterior surface and a posterior surface, and at least one of the anterior and posterior surfaces comprises an aspheric surface.

29. The method of claim 28, wherein the first artificial lens that has been positioned in the eye is configured to provide monofocal focusing.

30. The method of claim 28, wherein the first artificial lens has been positioned in a capsular bag.

31. The method of claim 28, wherein implanting the second artificial lens comprising implanting the second artificial lens posterior to an iris of the eye.

32. The method of claim 28, wherein the first artificial lens that has been positioned in the eye is configured to provide monofocal focusing.

33. The method of claim 28, wherein the anterior surface of the second artificial lens is convex.

34. The method of claim 28, wherein the posterior surface of the second artificial lens is concave.

35. The method of claim 34, wherein the posterior surface is concave such that the optic is meniscus shaped.

36. The method of claim 28, wherein at least one of the first and second lenses has 0 dioptric power.

37. The method of claim 28, wherein the transparent material comprises collamer.

38. The method of claim 28, wherein the transparent material comprises at least one of silicone, acrylic, and hydrogel.

39. The method of claim 28, wherein the anterior and posterior surfaces of the second artificial lens comprise aspheric surfaces.

40. The method of claim 28, wherein the anterior surface and the posterior surface each have a surface vertex, the optic having an optical axis through the surface vertices and a thickness along the optical axis that is in a range from about 100 micrometers to about 2 mm.

41. The method of claim 28, wherein implanting the second artificial lens into the eye comprises the one or more haptic portions contacting a sulcus of the eye with a pressure in a range from about 0.1 N to about 1.0 N.

42. The method of claim 28, wherein the anterior surface of the second artificial lens is substantially flat.

43. The method of claim 42, wherein the anterior surface of the second artificial lens is substantially flat such that the optic is plano-convex.

44. The method of claim 28, wherein implanting the second artificial lens comprises implanting the second artificial lens such that the posterior surface of the second artificial lens is substantially level with the plane of a sulcus of the eye.

45. The method of claim 28, wherein after implanting the second artificial lens, an iris of the eye rests in an approximately natural position.

46. The method of claim 28, wherein at least one surface of the first artificial lens and the second artificial lens includes a diffractive surface configured to divide incoming light to at least two independent foci.
